(19) United States
(12) Patent Application Publication
(10) Pub. No.: US 2025/0325774 A1
(43) Pub. Date: Oct. 23, 2025

(54) SYSTEMS, METHODS, AND APPARATUS FOR PRODUCING NITRIC OXIDE

(71) Applicant: VERO BIOTECH INC., Atlanta, GA (US)

(72) Inventors: Christopher Varga, Atlanta, GA (US); David Parrott, Cincinnati, OH (US); Simon Leclerc, Cincinnati, OH (US)

(21) Appl. No.: 19/186,911

(22) Filed: Apr. 23, 2025

Related U.S. Application Data
(60) Provisional application No. 63/637,663, filed on Apr. 23, 2024.

(51) Int. Cl. A61M 16/10; A61M 16/20; C01B 21/24

(52) U.S. Cl. CPC A61M 16/10; A61M 16/20; C01B 21/24; A61M 2202/0275

(57) ABSTRACT

Disclosed herein is a single-use container for forming a therapeutic amount of nitric oxide to be delivered by an apparatus to a subject, wherein the single-use container comprises: a housing defined by a proximal edge and a distal edge and comprising: a first chamber comprising N₂O₄, wherein the first chamber is sealed; a second chamber comprising an antioxidant material; and wherein the first chamber and the second chamber are positioned relative to each other such that when the single-use container is inserted into the apparatus and is activated, the first chamber is unsealed to allow the liquid N₂O₄ to be in fluid communication with the antioxidant material in the second chamber to produce nitric oxide.

### CROSS-REFERENCE TO RELATED APPLICATIONS

This application claims the benefit of U.S. Provisional Application No. 63/637,663, filed on Apr. 23, 2024, the contents of which are incorporated herein by reference in their entirety.

### FIELD

Some aspects described herein relate to a medical device and, more particularly, to systems and methods for producing and delivering a gas that includes nitric oxide.

### BACKGROUND

Some aspects described herein relate to the production of nitric oxide (NO), which is then typically delivered to a patient in a medical setting.

Nitric oxide is a vasodilator indicated to improve oxygenation and reduce the need for extracorporeal membrane oxygenation, particularly in term and near-term neonates with hypoxic respiratory failure associated with clinical or echocardiographic evidence of pulmonary hypertension in conjunction with ventilatory support. Low concentrations of inhaled nitric oxide can also prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, hyaline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism, idiopathic or primary pulmonary hypertension, and chronic hypoxia.

Inhaled nitric oxide therapy typically involves delivering nitric oxide in concentrations ranging from parts per billion to parts per million within a breathing gas, generally composed of air or oxygen-enriched air. This breathing gas may contain other components, such as anesthetic agents, nebulized liquids, or other gaseous components, and it is typically conveyed to a patient using either a mechanical or manual ventilation device. In some inhaled nitric oxide delivery systems, nitric oxide is provided within pressurized tanks, whereas in other systems, it may be generated on demand within the delivery system itself. One such system is described in U.S. Pat. No. 11,744,978, the content of which is incorporated herein in its entirety. In this approach, nitric oxide is produced through a chemical reaction between NO₂ gas and an antioxidant, where the NO₂ gas is generated via a phase change of liquid N₂O₄. In such systems, liquid N₂O₄ is typically housed in a pressure vessel with components required for reaction control (e.g., heating and cooling components), reactant mixing, and measurement, all of which are co-located with the reactants themselves. Although this is an effective approach, there is a need for a system wherein the reactants required to create nitric oxide gas for a patient are housed within a simple one-time-use component, and the components that are required to initiate, contain, measure, and control the reaction reside in a location where they can be used many times. This creates the need for novel packaging, geometries, and orientations of reactants, as well as novel loading, activation, and ejection mechanisms.

This need and all other needs are at least partially addressed by this disclosure.

### SUMMARY

The present disclosure is directed to a single-use container for forming a therapeutic amount of nitric oxide to be delivered by an apparatus to a subject, wherein the single-use container comprises: a housing defined by a proximal edge and a distal edge and comprising: a first chamber comprising N₂O₄, wherein the first chamber is sealed; a second chamber comprising an antioxidant material; and wherein the first chamber and the second chamber are positioned relative to each other such that when the single-use container is inserted into the apparatus and is activated, the first chamber is unsealed to allow the N₂O₄ to be in fluid communication with the antioxidant material in the second chamber to produce nitric oxide.

In still further aspects, the disclosure is directed to an apparatus comprising: an anvil positioned within a receptacle of the apparatus, wherein the receptacle defines a space configured to receive the single-use container of any of the examples herein, wherein the anvil is engageable with at least the proximal edge of the single-use container.

Still further disclosed herein is a system comprising: any of the disclosed herein single-use containers and/or any of the disclosed herein apparatuses.

Additional advantages will be set forth in part in the description that follows, and in part will be obvious from the description or can be learned by practice of the aspects described below. The advantages described below will be realized and attained by means of the chemical compositions, methods, and combinations thereof, particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 is a schematic of an exemplary system according to one aspect of the disclosure.

FIG. 2 is a schematic of an exemplary system according to one aspect of the disclosure.

FIG. 3 is a schematic of an exemplary system according to one aspect of the disclosure.

FIG. 4 is a schematic of an exemplary system according to one aspect of the disclosure.

FIG. 5 is a schematic of an exemplary system according to one aspect of the disclosure.

FIG. 6 is a schematic of an exemplary system according to one aspect of the disclosure.

FIGS. 7A-7I show various exemplary systems according to one aspect of the disclosure.

FIG. 8 is a schematic of an exemplary system according to one aspect of the disclosure.

FIG. 9 shows a side and a top view schematic of an exemplary single-use container before it is engaged with an exemplary anvil of an apparatus according to one aspect of the disclosure.

FIGS. 10A-10D are side-view schematics of an exemplary single-use container engaged with an exemplary anvil of an apparatus at different steps of delivering nitric oxide according to one aspect of the disclosure. FIG. 10E shows a top view schematic of an exemplary single-use container engaged with an exemplary anvil of an apparatus after the deactivation of the single-use container. FIG. 10F shows a side view schematics after disengaging the single-use container from the anvil and removal of the single-use container from the apparatus.

FIGS. 11A-11J show various views of the engagement of the single-use container within the apparatus.

FIGS. 12A-12F show various steps of operation of the exemplary system according to some aspects of the disclosure.

FIGS. 13A-13B depict a schematic of an exemplary single-use container according to some aspects of the disclosure.

FIGS. 14A-14C depict a closer view of an exemplary part of the exemplary single-use container of FIGS. 13A-13B.

FIG. 15 depicts a closer view of an exemplary part of the exemplary single-use container of FIGS. 13A-13B.

FIG. 16 depicts a closer view of an exemplary part of the exemplary single-use container of FIGS. 13A-13B.

FIG. 17 depicts a closer view of an exemplary part of the exemplary single-use container of FIGS. 13A-13B.

FIG. 18 depicts various steps of operation of the exemplary single-use container of FIGS. 13A-13B.

FIG. 19 depicts a disengagement step of the exemplary single-use container of FIGS. 13A-13B at the end of the operation.

FIGS. 20A-20C depict an exemplary flow-directing unit connected to the first chamber according to one aspect of the disclosure. FIGS. 21A-21B depict an exemplary single-use using the flow-directing unit and the first chamber shown in FIGS. 20A-20C according to one aspect of the disclosure.

FIGS. 22A-22C depict an exemplary flow-directing unit connected to the first chamber according to one aspect of the disclosure.

FIGS. 23A-23C depict an exemplary single-use container comprising the flow-directing unit and the first chamber shown in FIGS. 22A-22C according to one aspect of the disclosure.

FIG. 24 depicts exemplary needle configurations.

FIGS. 25A-25C depict an exemplary flow-directing unit connected to the first chamber according to one aspect of the disclosure.

FIGS. 26A-26E depict an exemplary single-use using the flow-directing unit and the first chamber shown in FIGS. 25A-25C according to one aspect of the disclosure.

FIG. 27 depicts an exemplary receptacle containing the single-use container of FIGS. 25A-26E and an exemplary anvil in one aspect of the disclosure.

FIG. 28 depicts exemplary steps of using the single-use container in the apparatus according to one aspect of the disclosure.

FIGS. 29A-29D depict an exemplary flow-directing unit connected to the first chamber according to one aspect of the disclosure.

FIGS. 30A-30D depict an exemplary receptacle containing the single-use container of FIGS. 29A-29D and an exemplary anvil in one aspect of the disclosure.

FIG. 31 depicts an exemplary receptacle containing the single-use container of FIGS. 29A-30D and an exemplary anvil in one aspect of the disclosure

FIGS. 32A-32D depict an exemplary flow-directing unit connected to the first chamber according to one aspect of the disclosure.

FIGS. 33A-33F depict various views of the exemplary single-use container according to one aspect of the disclosure.

FIGS. 34A-34G depict exemplary views of an exemplary valve.

FIG. 35 depicts a photograph of an exemplary single-use container.

FIGS. 36A-36F depict an assembled view (FIG. 36A) and exploded views (FIGS. 36B-36F) of the single-use container according to some aspects of the disclosure.

FIGS. 37A-37B depict a first diagonal cutaway view (FIG. 37A) and a second diagonal cutaway view (FIG. 37B) of the single-use container according to some aspects of the disclosure.

The accompanying figures, which are incorporated in and constitute a part of this specification, illustrate several aspects described below.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise. Thus, for example, a reference to “a single-use unit” includes not only one but also two or more such units, and a reference to “an apparatus” includes not only one but also two or more such apparatuses and the like.

Throughout the description and claims of this specification, the word “comprise” and other forms of the word, such as “comprising” and “comprises,” are open, non-limiting terms and mean “including but not limited to,” and are not intended to exclude, for example, other additives, segments, integers, or steps. Furthermore, it is to be understood that the terms “comprise,” “comprising,” and “comprises” as they relate to various aspects, elements, and features of the disclosed invention also include the more limited aspects of “consisting essentially of” and “consisting of.”

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalents thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The expressions “ambient temperature” and “room temperature” as used herein are understood in the art and refer generally to a temperature from 20° C. to 35° C.

All disclosed values also include values that fall within a ±10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase ‘x, y, z, or greater’ should be interpreted to include the specific ranges of ‘x,’ ‘y,’ ‘z,’ ‘about x,’ ‘about y,’ and ‘about z’ as well as the ranges of ‘greater than x,’ greater than y,′ ‘greater than z,’ or ‘greater than about x,’ greater than about y,′ ‘greater than about z.’ In addition, the phrase “‘x’ to ‘y’,” where ‘x’ and ‘y’ are numerical values, also includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges, as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given, and exemplary values are provided, it is understood that any ranges can be formed between any exemplary values within the broadest range. For example, if individual numbers 1, 2, 3, 4, 5, 6, 7, etc. are disclosed, then the ranges 1-7, 2-7, 3-7, 4-7, 5-7, 6-7, 1-6, 1-5, 1-4, 1-3, 1-2, 2-6, 2-5, etc. are also disclosed.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “and/or” includes any and all combinations of one or more of the associated listed items.

It will be understood that the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can, in some aspects, refer to at least 90%, at least 95%, at least 99%, or 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then 1% by weight, e.g., less than 0.5% by weight, less than 0.1% by weight, less than 0.05% by weight, or less than 0.01% by weight of the stated material, based on the total weight of the composition.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar importance.

As used herein, “treating” and “treatment” generally refer to obtaining a desired pharmacological or physiological effect. The effect can be, but does not necessarily have to be, prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” encompasses both inhibiting the disease, disorder, or condition, e.g., impeding its progression, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

Some aspects described herein relate to methods. It should be understood that such methods can be implemented using a computer. That is, where the method or other events are described herein, it should be understood that they may be performed by a computing device having a processor and a memory. Memory of a computing device is also referred to as a non-transitory computer-readable medium, which can include instructions or computer code for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also referred to as code) may be those designed and constructed for a specific purpose or purpose. Examples of non-transitory computer-readable media include, but are not limited to magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules, Read-Only Memory (ROM), Random-Access Memory (RAM) and/or the like. One or more processors can be communicatively coupled to the memory and operable to execute the code stored on the non-transitory processor-readable medium. Examples of processors include general purpose processors (e.g., CPUs), Graphical Processing Units, Field Programmable Gate Arrays (FPGAs), Application Specific Integrated Circuits (ASICs), Digital Signal Processor (DSPs), Programmable Logic Devices (PLDs), and the like. Examples of computer code include, but are not limited to, micro-code or micro-instructions, machine instructions, such as those produced by a compiler, code used to produce a web service, and files containing higher-level instructions that are executed by a computer using an interpreter. For example, aspects may be implemented using imperative programming languages (e.g., C, Fortran, etc.), functional programming languages (Haskell, Erlang, etc.), logical programming languages (e.g., Prolog), object-oriented programming languages (e.g., Java, C++, etc.), or other suitable programming languages and/or development tools. Additional examples of computer code include, but are not limited to, control signals, encrypted code, and compressed code.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein, and to the Figures and their previous and following description.

### Single-Use Container and Apparatus

In certain aspects, disclosed herein is a single-use container for forming a therapeutic amount of nitric oxide. Such a container is configured to be positioned within an apparatus that is configured to deliver the therapeutic amount of nitric oxide to a mammal. Any of the known in-the-art apparatuses that are compatible with the disclosed herein single-use container can be utilized. Some exemplary apparatuses are discussed below in more detail. It is understood that the term “mammal” refers to any mammal that requires a therapeutic amount of nitric oxide for whatever reason. In certain exemplary and unlimiting aspects, the mammal is a human patient. However, it is understood that the formed nitric oxide can be delivered to any other known mammal if needed.

It is understood that the single-use container disclosed herein can be used for medical purposes and, more specifically, for forming a desired amount of nitric oxide that can then be delivered to the subject. In aspects disclosed herein, the single-unit use container is utilized upon request and, by the end of the use, can be discarded and/or recycled if needed.

In still further aspects, the single-use container disclosed herein can comprise a housing defined by a proximal edge and a distal edge. In still further aspects, the housing can comprise a first chamber and a second chamber. In certain aspects, the first chamber comprises N₂O₄. In other aspects, the second chamber comprises an antioxidant material. In still further aspects, the first chamber is sealed. It is understood that the term “sealed” as used herein refers to the first chamber that can be fully sealed, or it can be partially sealed, or at least partially sealed. In still further aspects, the first chamber and the second chamber are positioned relative to each other such that when the single-use container is inserted into the apparatus and is activated, the first chamber can be unsealed to allow the N₂O₄ to be in fluid communication with the antioxidant material in the second chamber to produce nitric oxide. It is further understood that the unsealing process does not have to fully unseal the first chamber. In such aspects, the first chamber can be partially unsealed.

In certain aspects, the N₂O₄ is present as a liquid. In yet still further aspects, the liquid N₂O₄ can be in equilibrium with NO₂. Yet in still further aspects, the liquid N₂O₄ is in equilibrium with gaseous nitrogen dioxide (NO₂) or a gaseous mixture of dinitrogen tetroxide N₂O₄ and nitrogen dioxide (NO₂). In yet still further aspects, the first chamber can also comprise an amount of NO₂ or an amount of the gaseous mixture of dinitrogen tetroxide N₂O₄ and nitrogen dioxide (NO₂). In certain aspects, the first chamber can comprise a liquid phase and a gas space. In such exemplary and unlimiting aspects, the amount of NO₂ or an amount of the gaseous mixture of dinitrogen tetroxide N₂O₄ and nitrogen dioxide (NO₂) can be found in the gas space.

In still further aspects, it is understood that N₂O₄ can be present not as a pure liquid form. For example, N₂O₄ can be present as incorporated in additional media. For example, and without limitations, N₂O₄ can be present as a gel or any other matrix. It is understood that in such aspects, the media and/or matrix are not reactive towards N₂O₄. In such aspects, the media or matrix serves as a host of N₂O₄ to improve, for example, and without limitations, the safety and accessibility of the device. In such aspects, the matrix containing N₂O₄ can be positioned within the first chamber, and NO₂ can be formed during the activation of the first chamber. In still further aspects, the NO₂ formed during the activation of the first chamber can be positioned in the gas space of the first chamber.

In certain aspects, the antioxidant can be disposed within a solid matrix. In some aspects, the solid matrix can be a porous matrix. Yet, in other aspects, the solid matrix can take any form that allows for a high surface area and penetration of the antioxidant within the solid matrix. In still further aspects, it is understood, without being bound by any theory, that the high surface area of the solid matrix allows for a more efficient interaction between the antioxidant and N₂O₄ and/or NO₂, producing nitric oxide. In still further aspects, the antioxidant can be present without the solid matrix. For example, and without limitations, the antioxidant can be presented as a fluid. In certain aspects, the fluid can be stationary. Yet, in other aspects, the fluid can be continuously flowed through the second chamber to ensure a desired mixing between the antioxidant and the N₂O₄ and/or NO₂, producing nitric oxide. In further aspects, the second chamber can have agitation elements that allow for the desired mixing.

Antioxidants can comprise any known in the art antioxidants. In some aspects, the antioxidant can comprise ascorbic acid; alpha-, beta-, gamma-, or delta tocopherol; alpha-, beta-, gamma-, or delta-tocotrienol; polyphenols; beta-carotene; or a combination thereof. In still further aspects, the media comprising antioxidants can also comprise at least some amount of moisture.

In aspects where the solid matrix is present, such a solid matrix can comprise silica gel or other suitable high-surface area wettable material that is wetted, coated, or impregnated with the antioxidant. Nitrogen dioxide can react with an aqueous solution of the antioxidant to produce nitric oxide, following the following reactions:

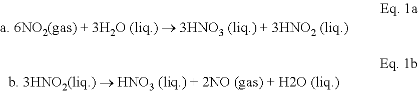

Yet, in other aspects, the NO can be formed according to Eq. 2a-2b:

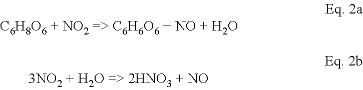

Ascorbic acid-wetted solid matrices (or other suitable antioxidant-containing materials) can be functionally similar to the media described in disclosed in U.S. Pat. Nos. 8,607,785, 8,944,049, 9,604,028, 10,926,054, 11,744,978, the contents of which are incorporated herein in their whole entirety.

Any of the antioxidants disclosed above can be present in an aqueous solution at concentrations ranging from 0 to 50 wt %, including exemplary values of 1 wt %, 5 wt %, 10 wt %, 15 wt %, 20 wt %, 25 wt %, 30 wt %, 35 wt %, 40 wt %, and 45 wt %. It is understood that antioxidants can be present in any amount that falls between any two disclosed above values, or they can be present in any range that can be formed by any two values that fall within the broadest range. For example, the antioxidant can be present in an amount of 1 wt % to 40 wt %, 1 wt % to 32 wt %, 5 wt % to 32 wt %, 15 wt % to 45 wt %, 1 wt % to 15 wt %, 10 wt % to 15 wt %, and so on. It may be desirable for the antioxidant concentration to be at or below the room temperature solubility limit because, at higher concentrations, the antioxidant may precipitate out, which could produce inconsistent nitric oxide conversion dynamics. It should be understood, however, that it may be possible to further increase the concentration of antioxidants by heating an antioxidant solution before wetting the solid matrix.

In still further aspects, the antioxidant is dispersed within a media comprising a support material having a specific surface area of 350 to 5000 m²/g, including exemplary values of 400 m²/g, 500 m²/g, 600 m²/g, 700 m²/g, 800 m²/g, 900 m²/g, 1000 m²/g, 1250 m²/g, 1500 m²/g, 1750 m²/g, 2000 m²/g, 2250 m²/g, 2500 m²/g, 2750 m²/g, 3000 m²/g, 3250 m²/g, 3500 m²/g, 3750 m²/g, 4000 m²/g, 4250 m²/g, 4500 m²/g, 4750 m²/g, and 4990 m²/g. It is understood that the specific surface area can be in any range formed between any two foregoing values. For example, and without limitations, the specific surface area can be 350 to 4500 m²/g, 400 to 5000 m²/g, 400 to 4000 m²/g, 500 to 5000 m²/g, 500 to 4000 m²/g, 500 to 3000 m²/g, or 500 to 2000 m²/g, and so on.

Yet in other aspects, the second chamber can comprise media that is described in U.S. Patent Application No. 63/573,175, the content of which is incorporated herein is all entirety.

In still further aspects, the second chamber can be filled with any of the disclosed herein or in U.S. Patent Application No. 63/573,175 antioxidant-containing media prior to sealing.

In yet still further aspects, the antioxidant-containing media can be compressed during assembly to form a precise packing, and this compression can be applied by the process of screwing the second chamber onto a flow-directing unit or screwing the flow-directing unit onto the second chamber as disclosed in more detail below. Yet in other aspects, the attachment of the second chamber to the flow-directing unit can be done by other than screwing methods. In certain aspects, the second chamber can be tightly inserted into the flow-directing unit and affixed to it. In yet still further aspects, the second chamber can be affixed to the first chamber and/or the flow-directing unit by any other known in the art methods to ensure, if needed, additional compression of the antioxidant media.

In still further aspects, the affixing of the second chamber allows to provide a sealed flow path between the first and second chambers. In still further aspects, the sealed flow path can be achieved by the presence of O-rings and retaining features such as a latch.

In certain aspects, the second chamber can also be sealed. In still further aspects, the second chamber can be partially sealed or at least partially sealed. In some aspects, the second chamber is unsealed when the single-use container is inserted into the apparatus. In yet other aspects, the second chamber can be partially unsealed.

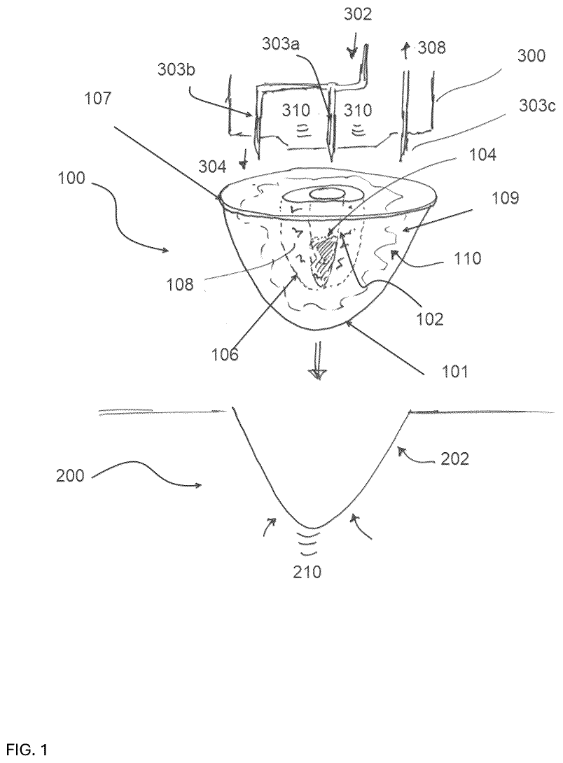

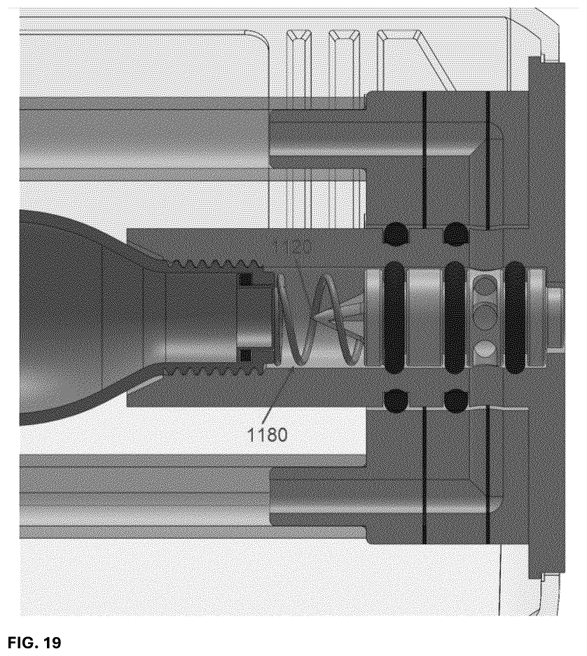

The exemplary and unlimiting single-use containers are shown in FIGS. 1-6, 8-10F, 13A-26E, 28-30D, and 32A-37B.

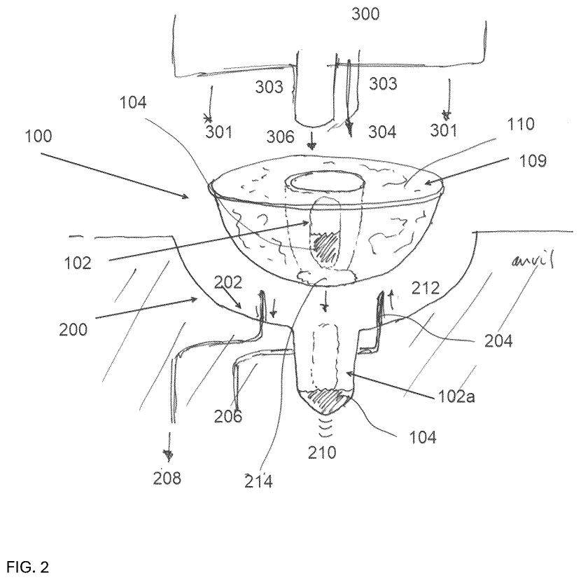

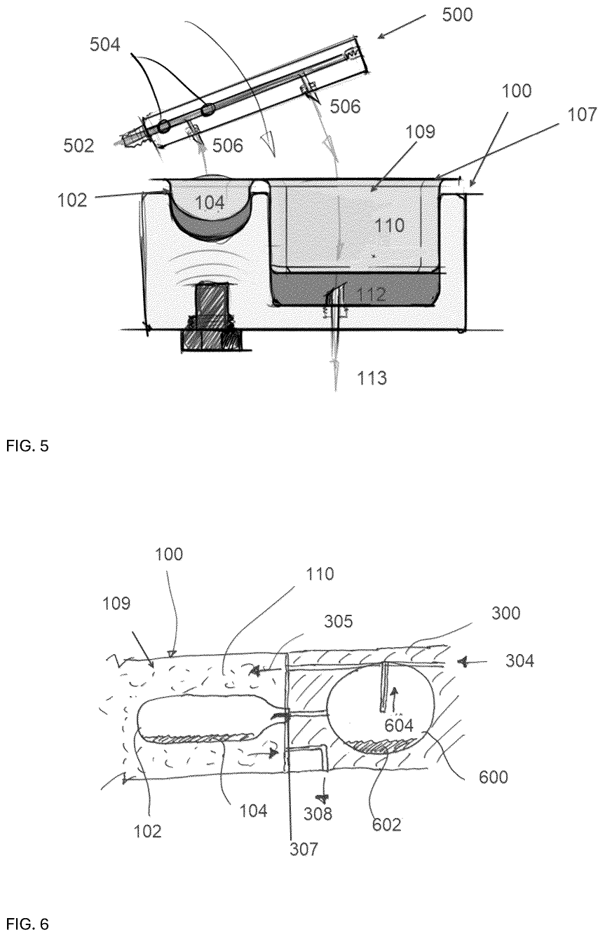

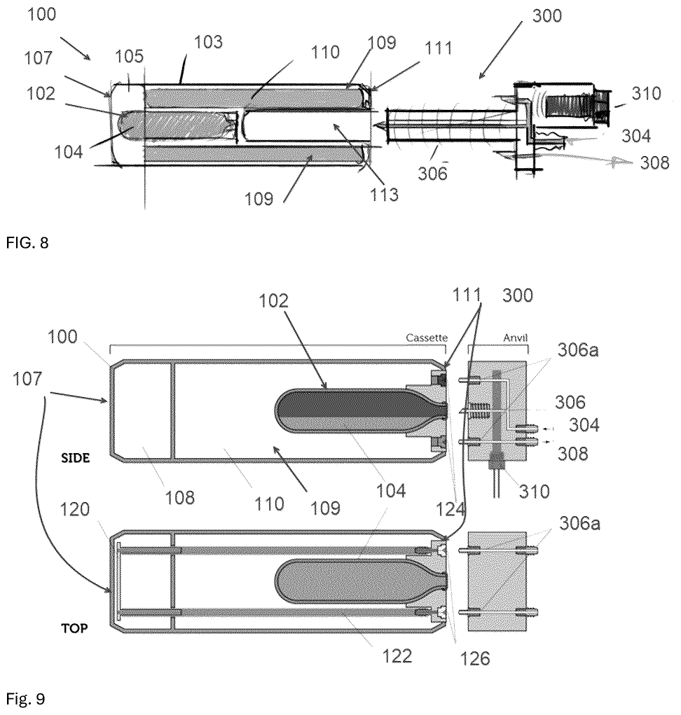

It is understood that the single-use container can have any desirable shape and form. The shape and form can be chosen to be ergonomic and to be capable of engaging with the delivery apparatus in the desired way. Referring to FIGS. 1 and 2, the single-use container 100 can have an exemplary shape of a pod. Again, it is understood that such a shape is exemplary and non-limiting. The single-use container 100 is shown in FIGS. 1 and 2 have a first chamber 102 that can comprise a liquid N₂O₄ 104 and a second chamber 109 that comprises an antioxidant 110. It can be seen that in this configuration, the first and second chambers are positioned concentrically to each other. For example, FIGS. 6 and 8 show configurations where the positioning of the first 102 and the second 109 chambers can be also assumed to be concentrical.

In still further aspects, the first chamber can be positioned within the second chamber. In such aspects, the two chambers do not have to share the same central axis. For example, the central axis of the first chamber can be offset from the central axis of the second chamber if desired.

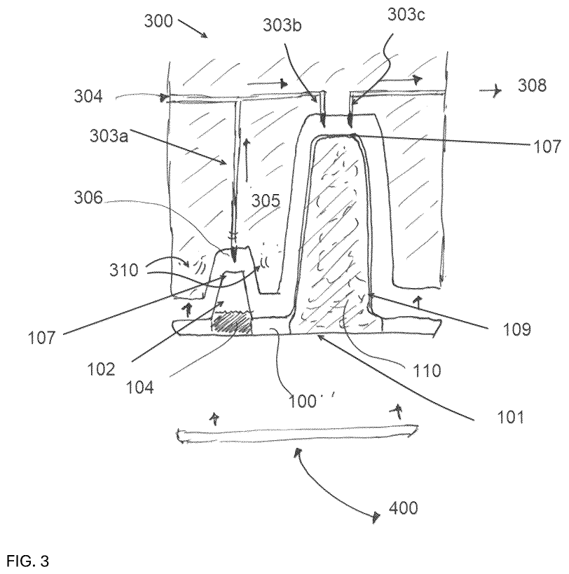

The exemplary single-use container 100, as shown in FIG. 3 has a different configuration. In this configuration, the first chamber 102 and the second chamber 109 are presented as two separated pods coupled to each other in series.

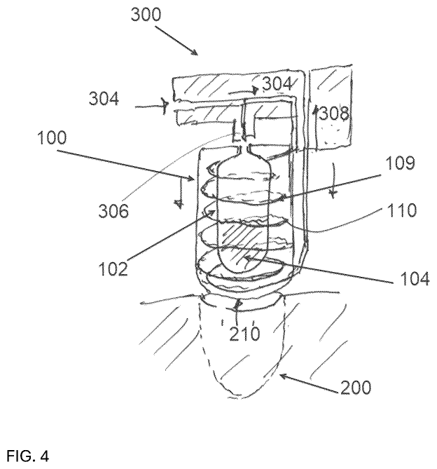

FIG. 4 shows an additional exemplary configuration of the single-use container 100. In this configuration, the first chamber 102 can be surrounded by a second chamber 109. In this exemplary aspect, the second chamber 109 can be presented as a tubing that wraps around the first chamber 102. In such an exemplary configuration, the second chamber can either have a solid matrix disposed within the tubing or can only comprise a granular, powder, gel, or fluid mixture of antioxidant 110 that is configured to come in contact with N₂O₄ and/or NO₂ to produce nitric oxide.

FIG. 5 shows a configuration similar to FIG. 3, where two chambers are positioned in series as two separate pods 102 and 109.

In still further aspects, and as will be shown below in more detail, when the unsealing of the first chamber occurs, the formation of the therapeutic amount of nitric oxide is initiated.

In still further aspects, the first chamber 102 is a pressure vessel. It is understood that the term “pressure vessel” as used herein refers to any vessel capable of containing (a liquid and/or gas and/or fluid and/or gel or solid) and/or withstanding pressure up to 1000 psi, up to 900 psi, up to 800 psi, up to 700 psi, up to 600 psi, or up to 500 psi. In still further aspects, the pressure vessel as described herein is capable of containing (a liquid and/or gas and/or fluid) and/or withstanding pressure of equal to or greater than 14 psi to 1000 psi, including exemplary values of 15 psi, 50 psi, 100 psi, 150 psi, 200 psi, 250 psi, 300 psi, 350 psi, 400 psi, 450 psi, 500 psi, 550 psi, 600 psi, 650 psi, 700 psi, 750 psi, 800 psi, 850 psi, 900 psi, and 950 psi. It is understood that any ranges between any two foregoing values or ranges formed by any two foregoing values can be formed. For example, and without limitations, the pressure vessel as described herein is capable of containing a liquid and/or gas and/or fluid and/or withstanding pressure of 15 psi to 600 psi, 15 psi to 500 psi, or 15 psi to 400 psi, or 100 psi to 900 psi, and so on. It is understood that the first chamber is made of a material that is capable of withstanding the disclosed above pressures and chemical conditions in which the chamber operates. In certain exemplary and limiting aspects, the first chamber can be made of stainless steel, aluminum, any conductive and inductive metals and alloys thereof, thermally conductive polymers, polymers, or any combination thereof. It is understood, however, that the materials used to form the first chamber are chosen to withstand the pressure and chemical conditions required for the first chamber operation. In certain aspects, the first chamber can also comprise an inductive element that can be heated if needed to initiate the desired reactions.

In still further aspects, the first chamber is directly filled with the N₂O₄ in a liquid form or with the N₂O₄ incorporated within a media. Yet in still further aspects, the first chamber can comprise an ampule filled with the N₂O₄. In certain and unlimiting aspects, the ampule can be positioned such that a proximal edge of the ampule is aligned with a proximal edge of the first chamber, and wherein the ampule is stationary within the first chamber.

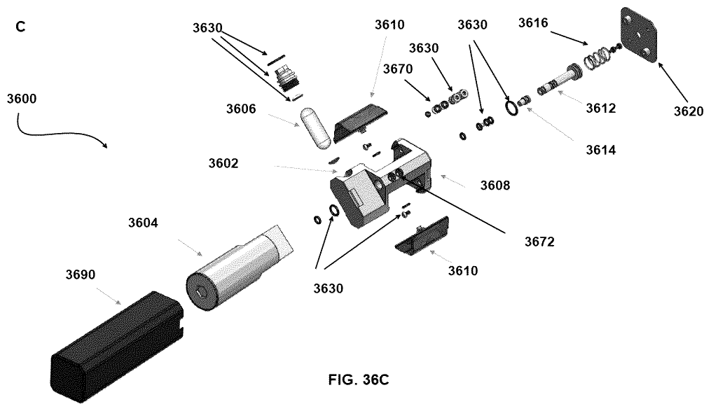

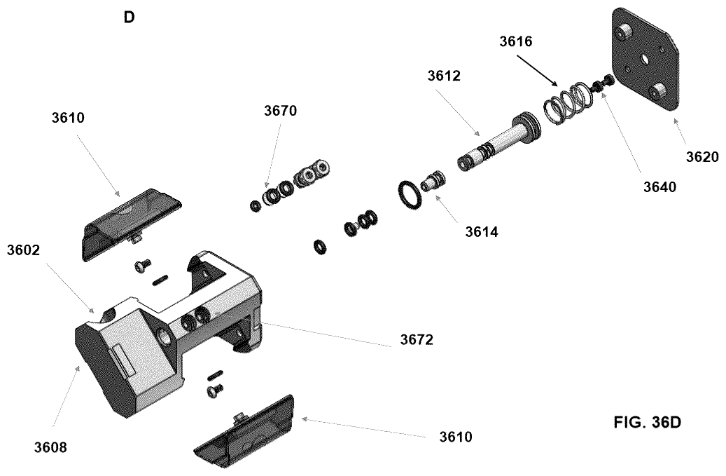

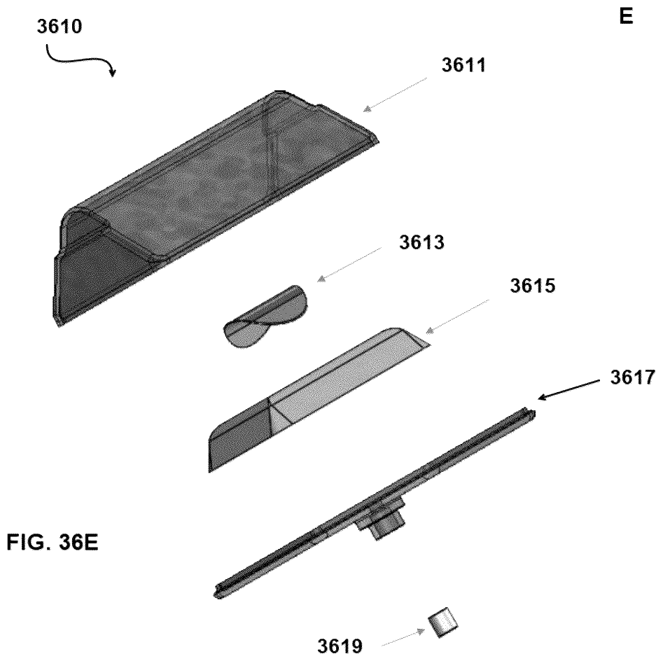

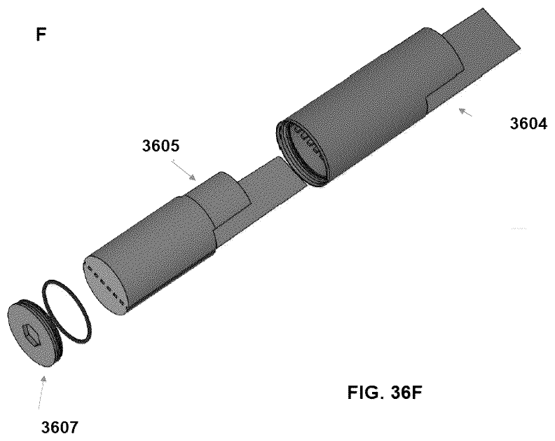

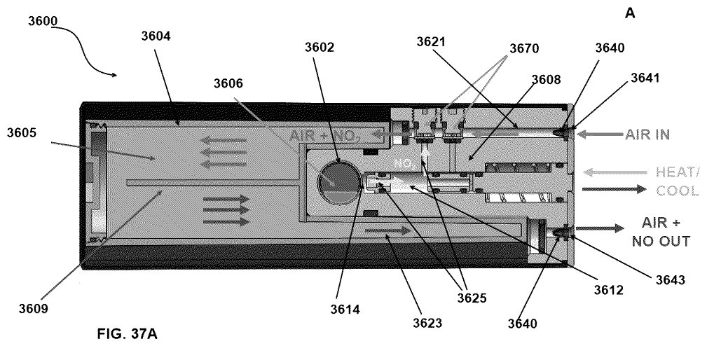

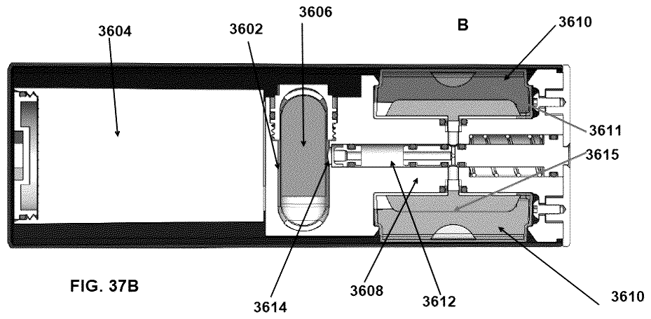

Yet in other exemplary and unlimiting aspects, the ampule can have any other positioning within the first chamber that suits the desired application. For example, as shown in FIGS. 36B-37B, and as discussed in more detail below, the ampule can be such that a middle portion of ampule 3606 is in parallel with a proximal edge (as it can be defined by a base plate 3620 in FIG. 36C), of the single-use container 3600, in other words, the ampule is positioned perpendicular to a main longitudinal axis L (FIG. 36A) of the single-use container.

In such aspects, the ampule can be a glass ampule, for example. Yet, in other aspects, the ampule can be positioned without the presence of the first chamber. Yet in still further aspects, the first chamber can be a receptacle or a mounting space that is configured to receive the ampule (FIGS. 36B-36F). In certain aspects, the ampule can be the first chamber. In such exemplary aspects, the breaking or piercing of the ampule results in directing its contents, e.g., the N₂O₄/NO₂, to a controlled/sealed path.

In still further aspects, and as shown herein, the housing of the single-use container can also comprise one or more inerting chambers. In such aspects, the one or more inerting chambers can be defined by a volume within the single-use container, or it can be a separate container that is coupled with other portions of the single-use container.

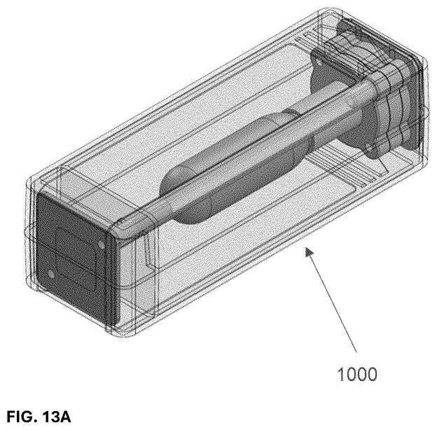

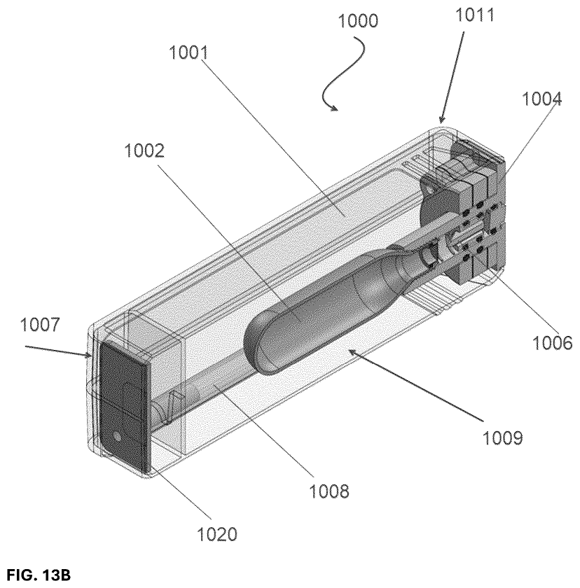

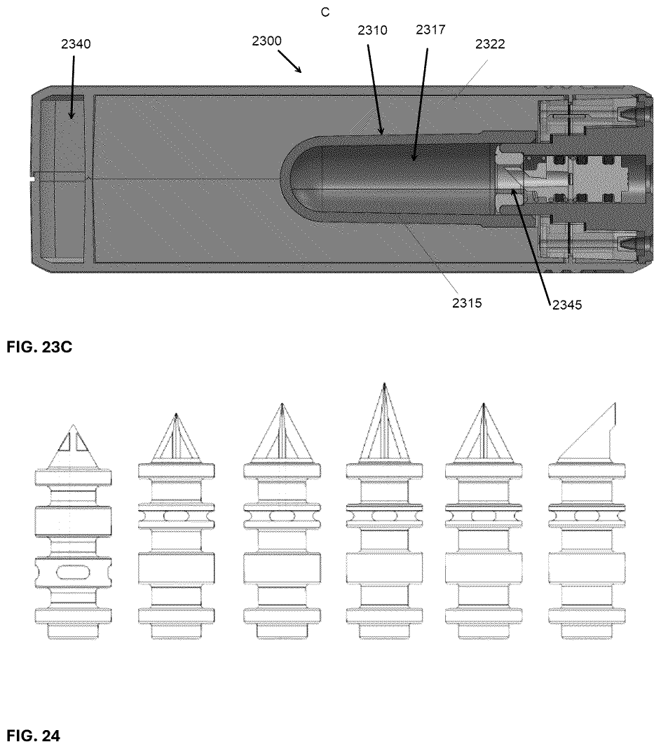

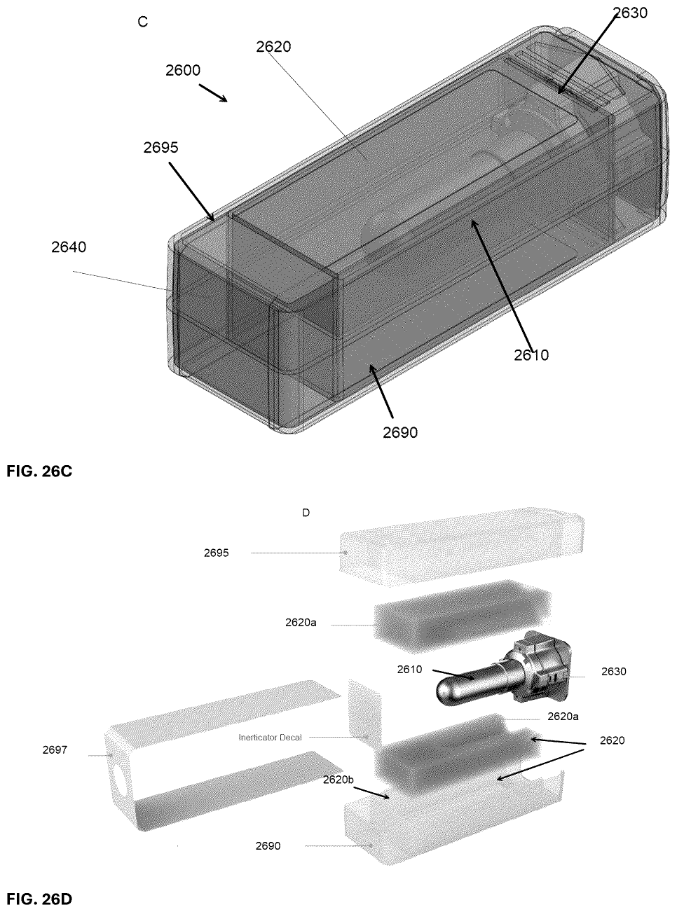

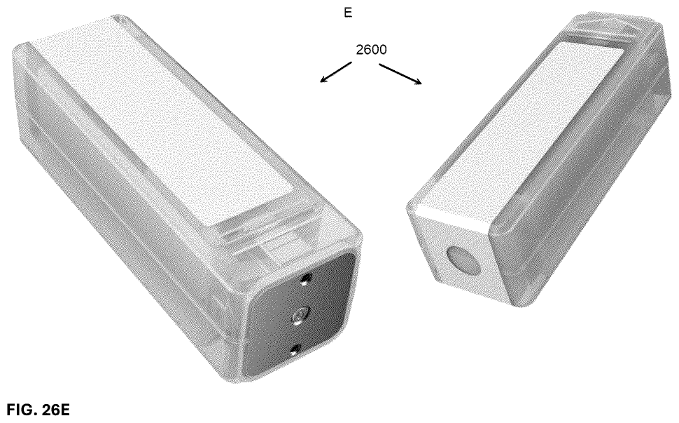

For example, the inerting chamber 106, as shown in FIG. 1, can also comprise an inerting material 108 that is configured to inert liquid N₂O₄ or gaseous NO₂ if exposed. In such exemplary and unlimiting aspects, the inerting chamber (it is understood that these two terms can be used interchangeably) can be positioned such that any inadvertent leak of N₂O₄ or NO₂ can be captured and contained within the inerting material. Yet, in other aspects, the inerting chamber can be utilized when the single-use container is consumed. In such aspects, any remaining N₂O₄ or NO₂ at the end of the use of the single-use container can be conveyed to and contained within the inerting chamber. The inerting chamber can have different positioning or configurations. For example, the inerting chamber 106 shown in FIG. 1 is concentric to the first chamber 102. While the inerting chamber 1020, as shown in FIG. 13B can be positioned in a distal portion of the single-use container. Similarly, the inerting chambers 2140 (FIG. 21A), 2340 (FIG. 23A), 2640 (FIG. 26A) are positioned in the distal portion of the single-use container.

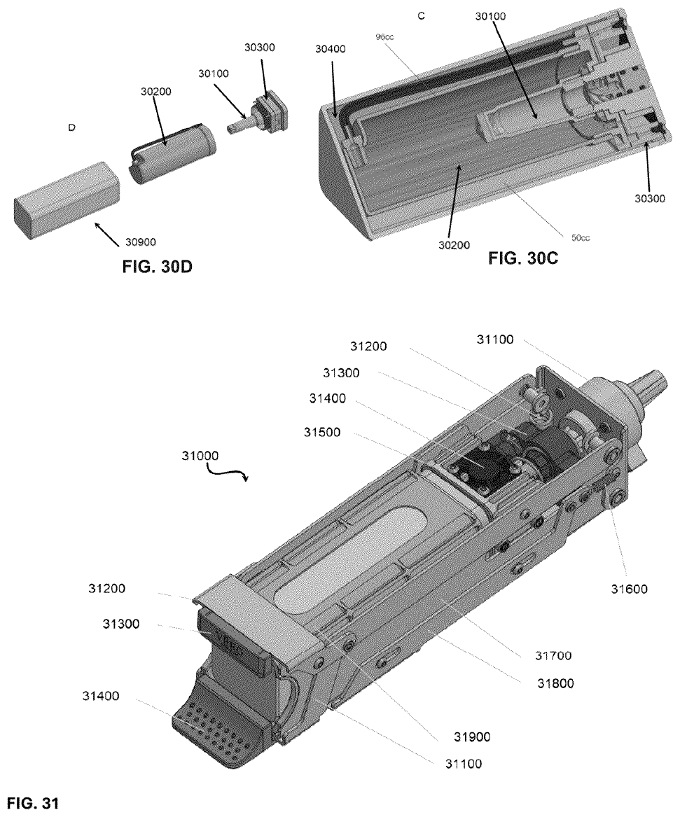

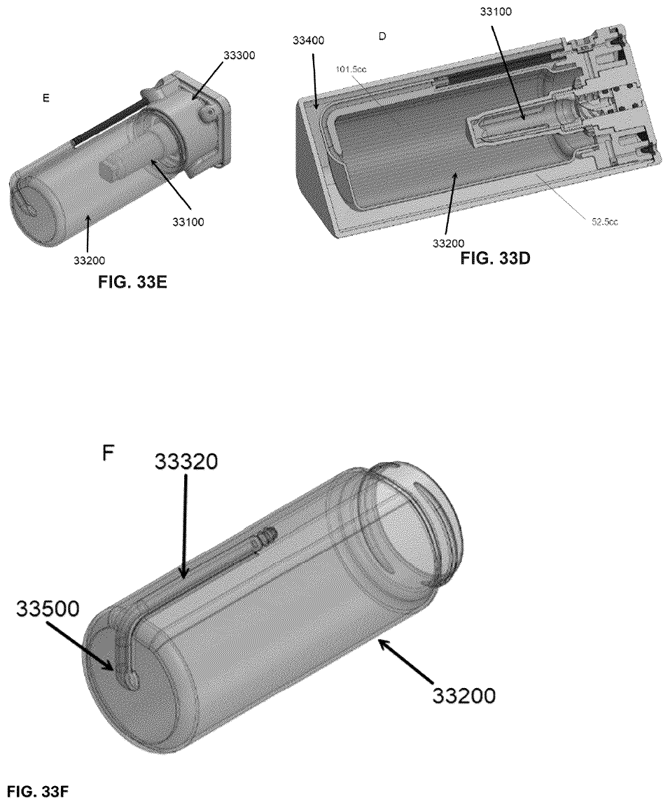

In yet other aspects, the inerting chamber can at least partially encompass the second chamber (3040 (FIG. 30A), 3340 (FIG. 33A, 33D).

In certain aspects, the one or more inerting chambers can be in fluid communication with the first chamber. Yet in still further aspects, the single-use container can comprise two inerting chambers 3610 (FIGS. 36B-36F) that are positioned in the proximal portion of the single-use container 3600. The exploded view of one of the two inerting chambers 3610 is shown in FIG. 36E. The inerting chamber can be defined, for example, and without limitations, by the housing comprising a plate 3617 that attaches the chamber to the proximal portion of the single-use container, the top cover (or housing) 3611 that hosts the inerting material 3615. In certain aspects, the inerting chamber can comprise a gas indicator 3613, for example, NO₂ gas. In still further aspects, the inerting chamber can comprise a filter 3619.

In certain aspects, the one or more inerting chambers can take the form of an open volume filled with the inerting material. In such exemplary and unlimiting aspects, the liquid N₂O₄ and NO₂ gas can be distributed throughout this inerting volume once directed by an appropriate mechanism. For example, and as disclosed in detail below, such an appropriate mechanism can be a flow-directing unit.

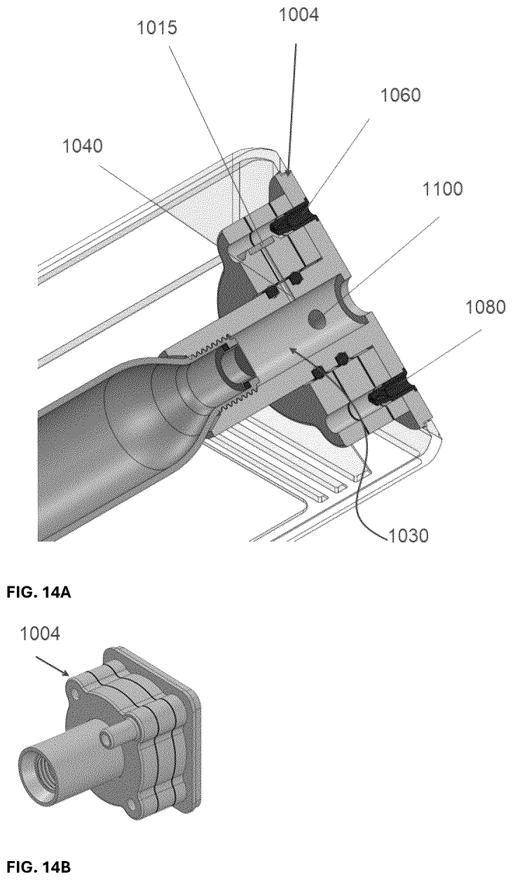

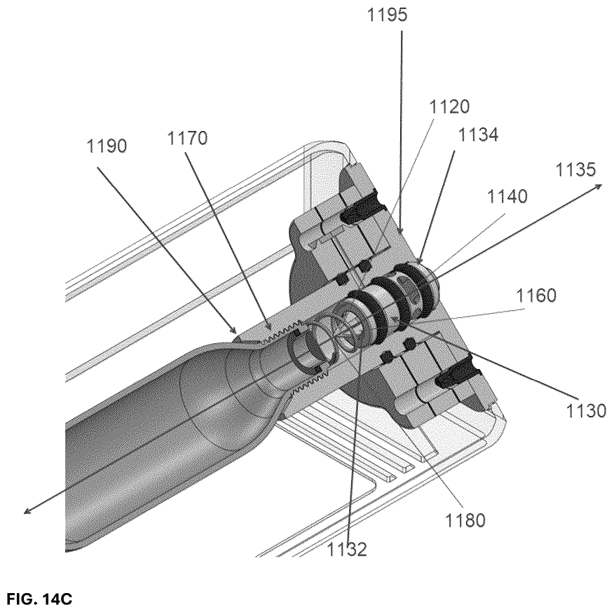

In certain exemplary and unlimiting aspects, the flow-directing unit is comprised of several components. In certain exemplary and unlimiting aspects, and as shown for example in FIGS. 14-19, the flow-directing unit comprises a base plate or first stage. In further aspects, the flow-directing unit can comprise a bed where a valve resides. In certain aspects, the valve can have a needle end and one or more side ports, inlet and outlet orifices with optional valves that are closed (or sealed), but openable (or unsealed) by mating surfaces in an anvil upon insertion of the single-use container into the mechanism and contact with the anvil.

In further aspects, the flow-directing unit can further comprise a second stage, which houses an orifice plate assembly that allows installation and removal of orifice plates with different size holes and also contains the flow paths that enable fluid communication between the first chamber and second chamber as well as the first chamber and the one or more inerting chambers depending upon the position of the spool valve. In still further exemplary and unlimiting aspects, the second stage connects with the first chamber through a threaded connection and mates to the second chamber through a bayonet-style threaded feature. In yet still further aspects, the second stage can also contain a flow path that connects the air/gas path from the first stage to the second chamber, as well as a flow path from the second chamber through the first stage to exit the single-use container. In still further aspects, the second stage can contain a frit/filter receptacle and frit/filter to prevent debris or particulates from exiting the second chamber.

In still further aspects, the valve is spring loaded to seal closed in a shipping or inerting position such that no gases leak from the single-use container to the ambient atmosphere. In still further aspects, the valve can contain a needle, for example, a beveled needle, comprising a side port that can pierce a metal burst disc (or a foil) as well as crack an ampule when advanced by the firing pin. In still further aspects, the valve can contain a frit or filter receptacle and frit/filter in its internal lumen to prevent debris or particulates from exiting the first chamber. Yet in other aspects, the distal end of the valve can comprise unsealing means that are different from the needle. In such aspects, the unsealing means 3614 (FIGS. 36B-36F) are configured to break ampule 3606. In this configuration, since the ampule is positioned perpendicularly to the valve, the breakage point can be somewhere in the middle of the ampule. The unsealing means 3614 does not have to have a sharp edge and can break the ampule based on the applied force. In still further aspects, the unsealing means can at least partially remain within the ampule wall after unsealing, thus preventing the shards or any other debris or particles from entering the internal lumen of the valve. In still further aspects, the unsealing means can also comprise a filter or a frit that can further prevent undesirable migration of the debris.

In still further aspects, the flow-directing unit can also contain a burst disc, which is captured and sealed upon the mating of the first chamber with the first stage.

The first chamber can also accommodate an ampule securing device, which holds an ampule of N₂O₄ in a fixed position within the first chamber to enable accurate breakage when contacted by the needle end of the valve and also secures the ampule during shipping, so it is not rattling around and is protected from accidental breakage.

A more detailed description is disclosed below and in the enclosed figures.

In other exemplary and unlimiting aspects, the inerting chamber can take the form of a tubular volume filled with the inerting materials. In other aspects, and as shown in FIGS. 36B-37B, the single-use container can comprise two inerting chambers that are positioned around the flow-directing unit in the proximal portion of the single-use container. It is understood that the liquid N₂O₄ and NO₂ gases can be directed to any of the disclosed herein inerting chambers through a flow path by the appropriate mechanism, such as the disclosed below flow-directing unit.

It is understood that the inerting material is capable of neutralizing or scrubbing any N₂O₄ or NO₂ that has not reacted or leaked. The inerting material can comprise any material capable of reacting with N₂O₄ or NO₂. For example, and without limitations, the inerting material can comprise a soda lime. In other exemplary and unlimiting aspects, the inerting material can comprise a Sodasorb, which is a mixture of sodium hydroxide and calcium oxide.

In certain aspects, the inerting material can comprise an indicator or a sensor showing the user that the inerting material was used. Such exemplary and unlimiting indicator 3613 is shown in FIG. 36E. For example, and without limitations, the inerting material can comprise a colorimetric indicator or sensor that is configured to change color when the inerting material interacts with N₂O₄ or NO₂. In certain aspects, the change in color can be observed by the operator. Yet, in still further aspects, the change in color can be determined by a detecting unit of the apparatus. In yet other aspects, the inerting can be detected chemically or electrically by the sensor. The detecting unit of the apparatus can then communicate with the control unit of the apparatus. In certain aspects, when the control unit of the apparatus receives a signal that the inerting material has been used, the control unit can provide a signal to the operator or a patient alerting them that the cassette or single-use container is no longer usable for therapy or of the leak or other reasons that the inerting material has been activated.

In certain aspects, as described herein and shown in the drawings, the first chamber 102 and the second chamber 109 can be separated from each other by a separating element, such that when the separating element is broken upon the single-use container activation, fluid communication between the first chamber and the second chamber is established. It is understood that the separating element can be any element known in the art that can be broken by an intermediary mechanism and/or initiation mechanism. It is further understood that the separating element does not have to have direct contact with either the first chamber or the second chamber. For example, the separating element can be seals that are unsealed by unsealing elements (such as piercing elements) of the apparatus, which is discussed in detail below.

It is understood that the single-use container can have any of the configurations disclosed herein and shown in the enclosed drawings. It is further understood that the first 102 and the second 109 chambers can be positioned anywhere within the housing, depending on the desired application and the apparatus that the single-use container is interacting with. As mentioned above, the first chamber 102 and second chamber 109 can be positioned concentrically (FIG. 1, 2, 4, 6, 8-10F, 12A-19) or in series (FIGS. 3 and 5). In certain aspects, the housing can comprise a secondary chamber 102a (FIG. 2) that can be fluidically connected with the first chamber 102. In such aspects, if desired, the liquid N₂O₄ can be released into the secondary chamber 102a. In such configurations, the formed NO can be delivered by different paths than the one disclosed in FIG. 1, depending on the specific apparatus that is used.

In still further aspects, the first chamber 102 can be positioned adjacent to a distal end 107 of housing 103 of the single-use container 100, as shown in FIG. 8. The housing can, in some aspects, be transparent, yet in other aspects, it can be opaque. It is understood that the housing itself can be formed from any materials suitable for the desired application. In still further aspects, in this configuration, the housing can have a space 113 configured to receive a matching portion of the apparatus, for example, an anvil 306, as described in more detail below. Yet in other aspects, as, for example, shown in FIG. 9, the first chamber 102 can be positioned in the proximal end 111 of the single-use container 100.

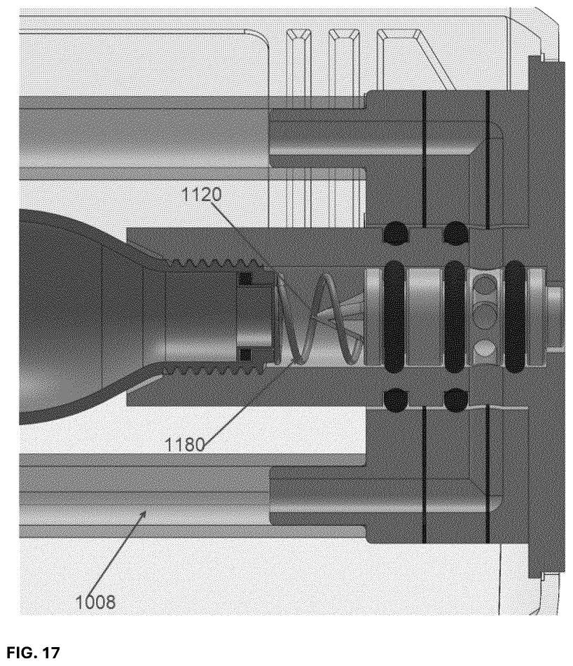

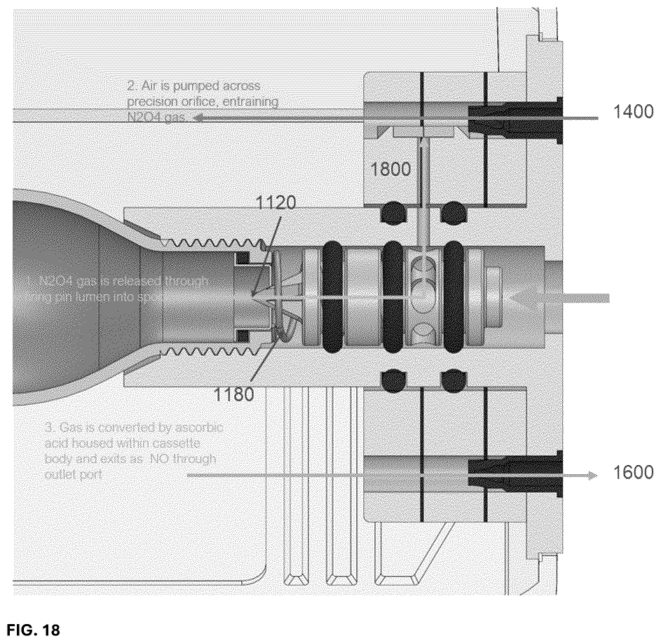

A single-use container 1000, according to an additional aspect, is shown in FIGS. 13A-16. A cross-sectional view of container 1000 is shown in FIG. 13B. The housing 1001 of container 1000 comprises a first chamber 1002 and a second chamber 1009 disposed around the first chamber. The housing can further comprise an inerting path or inerting tubing 1008 that is configured to convey and/or collect the remaining liquid N₂O₄ and/or NO₂ and to deliver it to inerting chamber 1020. Additional view of the container can be seen in FIGS. 17-19. In FIG. 18, it also shows air pumped from inlet 1400 and across a precision orifice and entering N₂O₄ gas, where the gas is converted by the antioxidant housed within the container, exiting as formed NO through outlet 1600.

In still further aspects, a proximal portion 1011 of the housing comprises a flow-directing unit 1004 (FIG. 13B). In such exemplary aspects, the flow-directing unit 1004 can be fluidically connected to the first chamber 1002 and the second chamber 1009 and is configured to form and/or interrupt a flow path between the first chamber 1002 and the second chamber 1009 as shown in FIG. 14A. The flow-directing unit 1004 is further fluidically connected through the orifice 1100 to the inerting chamber 1020 through the inerting tube 1008 and is configured to form and/or interrupt a flow path between the first chamber and the inerting chamber. It is understood that the inerting tubing can be an inerting flow path that is a part of the second chamber that communicates with the inerting chamber.

The flow-directing unit 1004 further comprises an inlet port 1060 that is configured to receive air from a matching portion of the apparatus, as is described in more detail below. The flow-directing unit 1004 further comprises an outlet port 1080 that is configured to match with a portion of the apparatus as it is described in more detail below and to transfer formed gas from the single-use unit to a patient. The flow-directing unit 1004 can further comprise an orifice plate 1015 having an orifice size of 1 micron to 100 microns that allows the fluid connection between the gas passage 1040 to the second chamber and the second chamber 1009. In still further aspects, the orifice plate can have an orifice size of 1 micron to 100 microns, including exemplary values of 3, 5, 10, 15, 20, 25, 30, 40, 50, 60, 70, 80, and 90 microns. It is understood that any ranges between any two foregoing values can be formed. For example, and without limitations, the orifice can have a size of 1 to 90 microns, 1 to 70 microns, 1 to 50 microns, 1 to 20 microns, 1 to 10 microns, 5 to 100 microns, 5 to 90 microns, 5 to 50 microns, 5 to 30 microns, and so on. The size of the orifice can assist in determining the flow of nitric oxide as desired. In such aspects, the flow-directing unit can be constructed such that it can accept orifice plates with different orifice sizes—this capability will create the opportunity for single-use containers that support specific dosage ranges—e.g., micro-dosing, standard, high-dose, etc.

A general view of the flow-directing unit 1004, along with a more detailed view of the unit, is shown in FIGS. 14B-14C. The flow-directing unit further comprises a valve 1130. The valve comprises a proximal end 1134 and a distal end 1132 and is configured to move along a main axis 1135 of the single-use container. It is understood that the valve is positioned within bed 1030 (FIG. 14A), configured to host the valve. In still further aspects, the valve is configured to regulate the flow path between the first chamber and the second chamber and/or the first chamber and the inerting chamber, depending on the valve position. The flow path is regulated by the positioning of the gas outlet 1140 within the valve. It is understood that any known in the art valves capable of controlling the flow as described can be utilized. In some exemplary and unlimiting aspects, the valve is a spool valve.

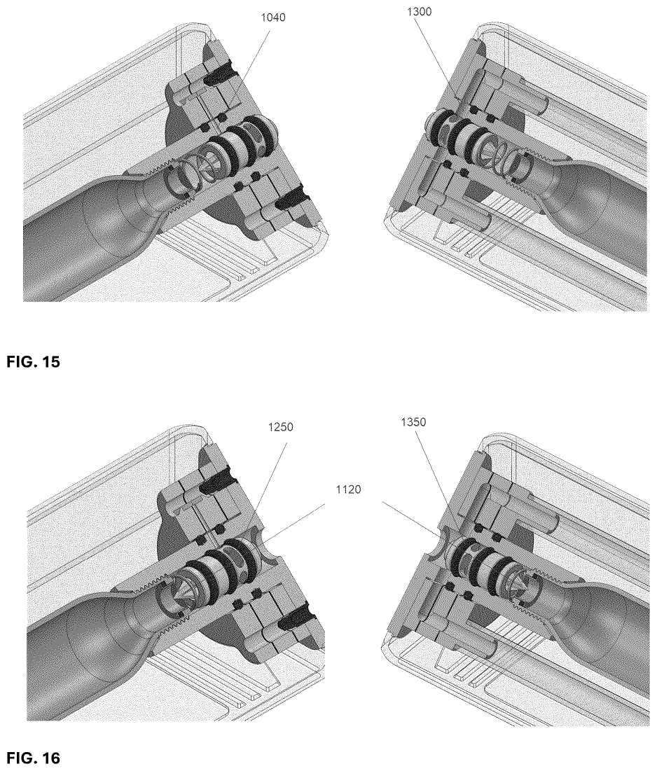

In certain aspects, before the single-use container is used, the valve is positioned such that if the first chamber is undesirably unsealed, a flow path 1300 is formed between the first chamber and the inerting chamber (FIG. 15, right side). Similarly, when the single-use container is fully utilized, the valve is positioned such that a flow path is formed between the first chamber and the inerting chamber to remove unreacted N₂O₄ and/or NO₂. It can be seen that when the single-use container is not in use, passage 1040 to the second chamber is sealed and has no flow from the valve (FIG. 15, left side).

In still further aspects, and as shown in FIG. 14C, valve 1130 comprises a needle 1120 that is positioned within the body of the valve and is configured to unseal the first chamber when activated within the apparatus. In still further aspects, the needle 1120 defines the distal end 1132 of the valve 1130. In still further aspects, the valve 1130 is a spring-loaded valve. In such aspects, the valve is coupled with spring 1180 that is configured to activate the needle 1120 to unseal the first chamber and/or also return the firing pin back at the end of the use.

In still further aspects, the valve comprises an internal lumen extending from the proximal end 1134 to the distal end 1132 of the valve. The internal lumen has a first edge and a second edge, wherein the first edge is sealed on the proximal edge of the single-use container (not shown), and the second edge 1160 defines a distal edge (or a sharp edge) of the needle firing pin 1120. It is understood that in certain aspects, the flow paths within the valve can also comprise a pledget or frit to filter the N₂O₄/NO₂ fluid that is conveyed to the micro orifice plate or capillary.

In still further aspects, a distal edge portion 1190 of the flow-directing unit comprises a mounting space 1170 configured to receive the first chamber. In certain aspects, this mounting space is configured to be heated. It is understood that any known in the art methods of heating can be utilized. In certain aspects, the mounting space can be inductively heated or heated by conduction, radiation, or by any combination thereof.

In still further aspects, and as disclosed above, the first chamber comprises a liquid N₂O₄ or N₂O₄ comprised in a solid matrix. In still further aspects, the first chamber can also comprise NO₂. When the single-use container is inserted into the apparatus and activated, the valve is moved toward the first chamber such that the needle 1120 penetrates and unseals it, allowing the release of N₂O₄/NO₂. In such aspects, the valve is positioned so that the valve's gas outlet 1250 is open towards the second chamber, creating the desired fluidic path while keeping the path 1350 to the inerting chamber closed (FIG. 16). It is also understood that the needle used in this disclosure is also configured to unseal an ampule if the ampule is used as a N₂O container.

In still further aspects, any of the disclosed herein single-use containers are configured to be received by an apparatus. The apparatus can comprise a receptacle. Such an exemplary receptacle 200 is shown in FIG. 1-2, 4, or 400 in FIG. 3. In certain aspects, the receptacle has a mating surface 202 that is in contact with a distal end 101 of the single-use container and can also be heated by a heating element 210. Again, it is understood that any known in the art heating elements can be used.

The apparatus can further comprise an anvil. For example, FIGS. 1 and 2 show an anvil 300 that is configured to engage with at least a portion of the proximal edge 107 of the single-use container. In FIG. 3 wherein the first 102 and the second 109 chambers are disposed in series, each of the chambers has its own proximal edge 107 that can engage with the anvil 300. In such an exemplary configuration, the single-use container can be held by a plate 400, for example.

A different configuration of engagement is shown in FIG. 4. In this configuration, the second chamber 109 wraps around the first chamber 102, is positioned into a receptacle 200 of the apparatus, and is engaged with the anvil 300. In this exemplary and unlimiting aspect, the air/gas flow can enter from the left of the pod, and then it can be delivered, for example, through an orifice plate similar to the one disclosed above. Then, that flow of air/gas plus NO₂ can continue and can connect directly at the right side to the tubing that contains the antioxidant.

In the configuration shown in FIG. 5, the activation can be done by positioning anvil 500 on the proximal edge 107 of the single-use container 100 and engaging it.

In still further exemplary configurations, as shown in FIG. 6, the anvil 300 can comprise a secondary first chamber 600 that can be in fluid communication with the first chamber 102 after the single-use container 100 is inserted into the apparatus.

FIG. 8 shows a configuration where the single-use container has a space 113 configured to receive the anvil 300. In this specific exemplary aspect, a portion of the anvil 306 is configured to be inserted into space 113 and unseal the first chamber 102. In certain aspects, the portion of the anvil 306 can also be heated.

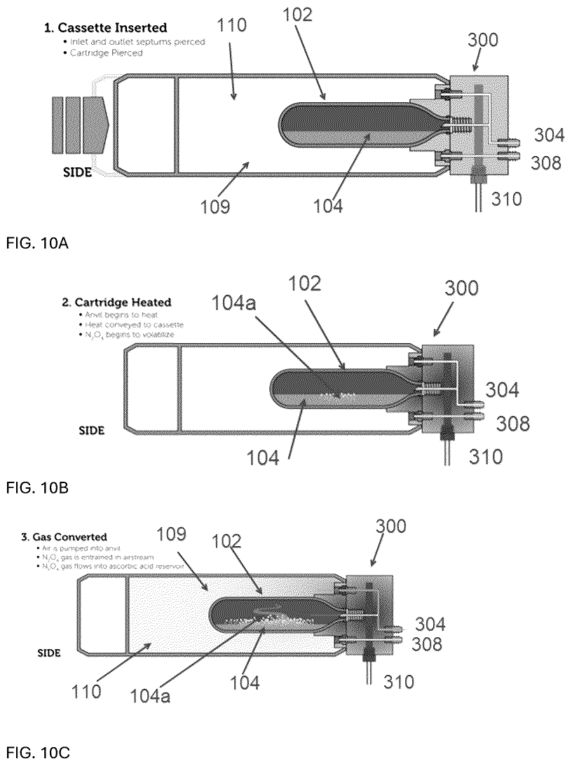

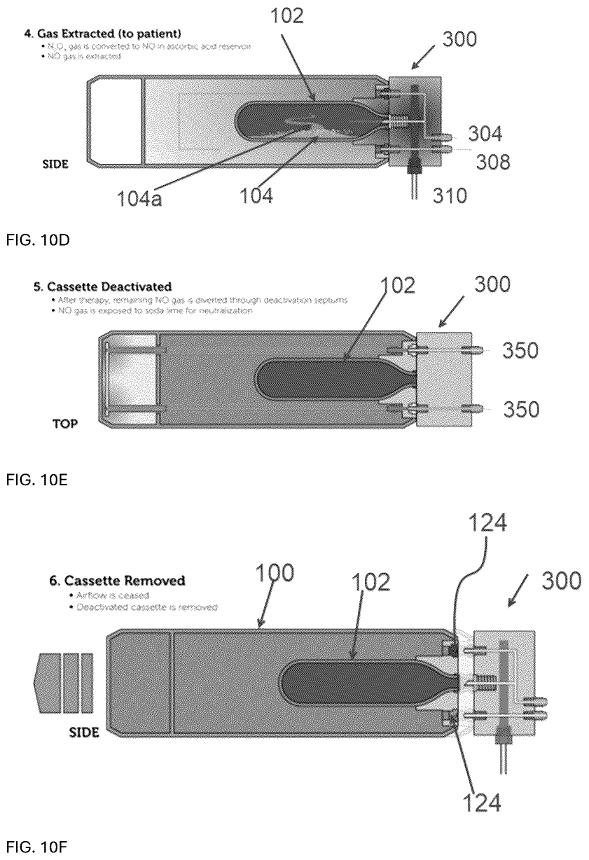

In FIGS. 9-10F, the proximal end 111 of the single-use container 100, is engaged with the anvil 300.

The single-use container is depicted in FIGS. 13A-19, when inserted into the apparatus, can also be engaged with a mating surface of the apparatus (not shown). Such a surface can be a portion of an additional anvil disposed within the apparatus or any other surface that can comprise mating elements that can engage with the single-use container.

In still further aspects, the apparatus is configured to activate the single-use container to form the nitric oxide.

It is understood that the way the single-use container is activated would depend on the configuration of the single-use container and the apparatus.

For example, as shown in FIG. 1, when anvil 300 is engaged with the single-use container 100, the proximal edge 107 interacts with one or more elements 303 of the anvil. Each of the one or more elements 303 comprises a lumen configured to create a fluidic path between the apparatus and the single-use container. In such aspects, one or more of the elements 303 are configured to unseal at least a portion of the first and/or second chambers. It is understood, however, that the unsealing occurs at a specific location by insertion of the one or more elements 303 into the designated chamber. The unsealing occurs between the chamber and the one or more elements but not between the chamber and the surrounding environment. In other words, the term unsealing, as used herein, refers to creating a fluidic path between the first and the second chambers and the apparatus using the lumens of the one or more elements 303a-303b. It is understood that the mere insertion of the one or more elements into the single-use container does not unseal the container itself to the surroundings. In fact, in some exemplary aspects, one or more elements are configured such that when they are inserted into the first and/or the second chambers, they form an additional seal between the chambers and the surrounding environment.

In still further aspects, the lumen of the one or more elements 303a-303b is configured to transport a gas to and from the desired chambers. For example, and without limitations, one or more of the 303a-303b units can deliver a gas mixture 302 from the apparatus to the first chamber. In such exemplary aspects, the gas mixture can comprise air, oxygen-enriched air, oxygen, nitrogen, or any combination thereof. The unsealing of the first chamber creates a fluid passage that allows the N₂O₄ present in the first chamber to be exposed to additional volume, enabling the liquid to undergo a phase change from liquid to gas and formation of NO₂ for conveyance 304 to the second chamber 109 comprising the antioxidant 110. The flow 304 can comprise a gas mixture delivered to the first chamber and the NO₂.

It is understood that the NO₂ formation process and the rate at which NO₂ flows through the first fluid passage can be controlled through the heating or cooling of the mating surfaces, which in turn heats or cools the N₂O₄/NO₂ mixture. Controlling the temperature can also allow control of the amount of NO₂ formed and, as a result, control the amount of NO formed in the single-use container. It is understood, and as described in this disclosure, the cooling, if present, can be achieved by any known in the art methods. For example, the cooling system can comprise fins, fans, and the like. In such exemplary and unlimited aspects, the fins and a fan can be activated to blow air across the fins. Yet, in other aspects, a Peltier-type cooling to extract heat from the process can be utilized.

In certain aspects, additional valves may be present to open and close communication between the N₂O₄ portion of the container and the one or more elements 303a-303b. Opening and closing of these valves enable control of the conveyance of NO₂ through the lumens. Once NO₂ is mixed into the gas mixture 302, the mixed flow passes into the antioxidant portion of the container. As the NO₂ gas mixture passes through the antioxidant, it reacts in a manner that converts NO₂ to NO through a series of reactions gas. The formed NO gas is then collected through lumen 303c and delivered as a NO mixture 308 to a patient. It is understood that in these exemplary aspects, the NO is conveyed to the patient as a mixture of NO and conveying gas described herein, wherein conveying gas can comprise air, oxygen-enriched air, nitrogen, or any combination thereof.

The concentration of NO within the mixed flow 308, which exits the antioxidant, is determined by the outflow rate of NO₂ from the first chamber, which initially contains liquid N₂O₄, as well as the flow rate and components of the conveyance gas 302, which the NO₂ mixes with.

FIG. 2 shows additional aspects, where the anvil 300 can push the first chamber 102 through the foil 214 into a secondary chamber 102a. Yet, in other aspects, only liquid N₂O₄ is pushed to the secondary chamber 102a. In this exemplary configuration, the conveyance gases are provided from the distal end of the single-use container. For example, the gas mixture 206 and NO₂ 212 interact with the antioxidant in the second chamber to form NO gas mixture 208 that is delivered to a patient.

FIG. 3 shows an exemplary configuration, where the one element 303a unseals the first chamber 102 and delivers a gas mixture 304 to the first chamber. Still further, a NO₂ gas is released, forming a conveyance gas 305 that delivers it to the second chamber through the element 303b. The NO gas 308 formed as a reaction of NO₂ with antioxidants is then delivered through lumen 303c to a patient.

As described above, some of the configurations can comprise an anvil comprising a secondary first chamber 600 comprising a liquid N₂O₄ 602 (FIG. 6). In this configuration, a gas mixture 304 passes through the secondary first chamber 600. The NO₂ gas formed in this chamber is then mixed with the gas mixture 304 to form NO₂/gas mixture 305 that contacts the antioxidant material 110 in the second chamber of the single-use container. It can be seen that there is fluidic communication 307 between the first chamber 102 and the secondary first chamber 600. The formed NO gas mixture 308 is then delivered to the patient.

In FIG. 8, the gas mixture 304 is delivered through anvil 306 to the first chamber, and then the NO₂/gas mixture comes into contact with the antioxidant material. The formed NO gas mixture 308 is then delivered to the patient.

FIG. 9 shows the coupling schematic between the exemplary single-use container 100 and the anvil 300. When the anvil 300 engages with the proximal end 111 of the container, the piercing element 306 unseals the first chamber 102, while piercing element 306a unseals activation septum 124. It is understood that in the exemplary aspects disclosed herein, under a spring force, the single-use container proximal surface comes into substantially intimate contact with the anvil to ensure efficient conductive heat transfer.

The gas mixture 304 is delivered through lumens for the piercing elements to the first chamber. The NO₂/gas mixture is then delivered to the second chamber 109 comprising an antioxidant 110. The formed NO gas mixture 308 is then conveyed to the patient. In certain aspects, the anvil portion of the apparatus is heated. The distal end of the container, an inerting chamber 108 comprising inerting material, is positioned to inactivate N₂O₄ and/or NO₂ in case of a leak or when the remaining N₂O₄ and/or NO₂ are left in the system at the end of the procedure. In certain exemplary and unlimiting aspects, the single-use container can also comprise a deactivation plate. In such exemplary and unlimiting aspects, the deactivation plate is configured to distribute the flow coming from the proximal end of the cassette to the inerting chamber.

FIGS. 10A-10F show various steps of use of the single-use container. In FIG. 10A, the single-use container 100 and anvil 300 are engaged, and the seals are pierced. The anvil is then heated with the use of heating element 310 (FIG. 10B), and NO₂ (104a) starts forming from the liquid N₂O₄ (104). The gas mixture 304 is delivered to the first chamber and is transferred to the second chamber 109 together with NO₂ (FIG. 10C). The formed NO (from the interaction of NO₂ with the antioxidant in the second chamber) gas mixture 308 is then delivered to the patient (FIG. 10D). At the end, the first chamber is substantially free of liquid N₂O₄ and NO₂. Whatever remaining amounts of N₂O₄ and NO₂ that are potentially still left in the system can be inactivated by the inerting material and dumping plate (FIG. 10E). FIG. 10F shows the disengagement of the single-use container from the anvil.

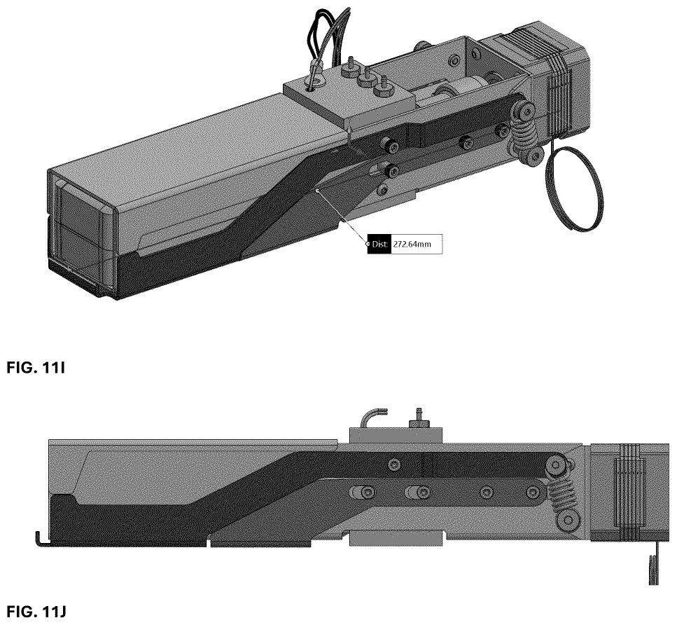

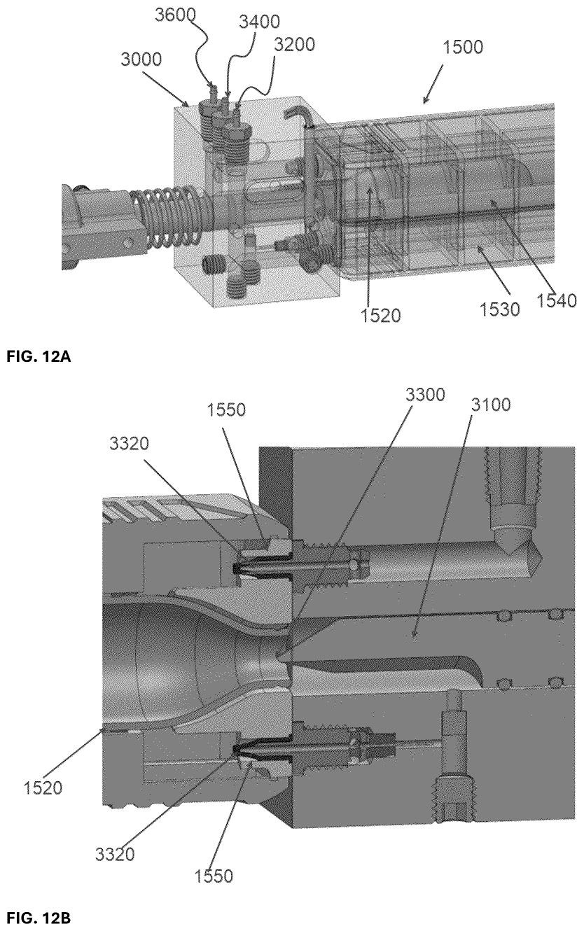

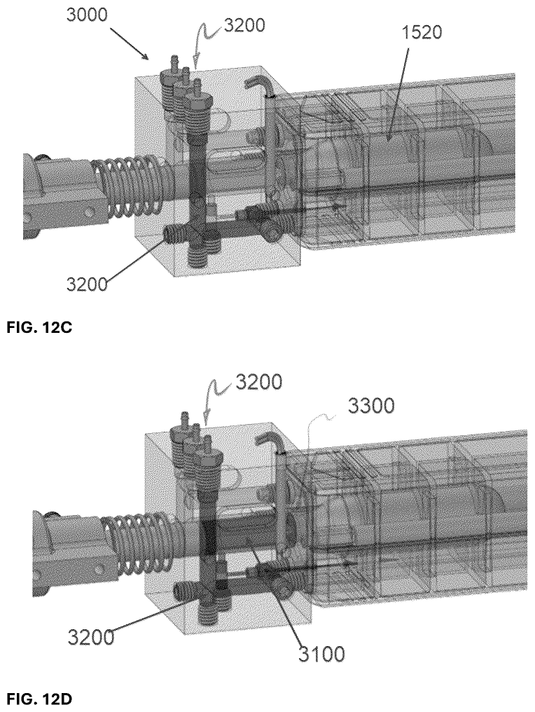

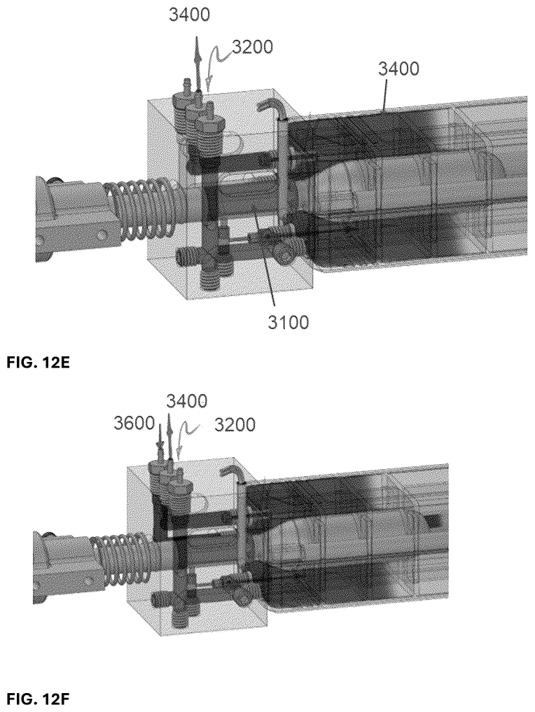

Additional aspects of the engagement between the anvil and the single-use container are shown in FIGS. 12A-12F. It can be seen that the single-use container 1550 comprises a first chamber 1520, the second chamber 1530, and the recycling tubes 1540. The anvil 3000 comprises fluidic paths 3200, 3400, and 3600 that can be utilized in various steps of the procedure. Upon engagement of the first chamber 1520 with the anvil, a firing pin 3300 present in the unsealing element 3100 unseals the first chamber while piercing elements 3320 unseal activation septum 1550 (FIG. 12B).

The gas mixture 3200 starts immediately flowing through the system, and when the first chamber is unsealed, NO₂ gas 3100 gets mixed with the gas mixture 3200 (FIG. 12C) to arrive at the second chamber. The formed NO gas, then 3400, is then conveyed to the patient. (FIG. 12E).

It is understood that the system that comprises the apparatus and the single-use container can comprise a plurality of valves configured to control the gas flow as desired. It is also understood that the system can also comprise any fittings and tubing that would provide for the desired flow rate and desired flow direction. In certain aspects, the dimensions of the tubing can be determined based on the desired flow. In certain aspects, the tubing is a capillary tube. In still further aspects, the system can comprise O-rings and any other fitting and sealing elements as needed.

To deactivate the unit, a downstream valve can be used to redirect the remaining gas 3600 back into the single-unit recycling (dump) chamber (FIG. 12F).

Referencing back to FIGS. 12A-19. When the single-use container 1000 is inserted into the apparatus (not shown), valve 1130 is activated such that the first chamber is unsealed and a flow path between the first chamber and the second chamber is formed. In still further aspects, when the single-use container is positioned within the receptacle, at least a portion of the receiving surface of the receptacle mates with at least a portion of the proximal edge of the housing such that the second chamber is unsealed at a second chamber inlet 1060 and a second chamber outlet 1080.

In still further aspects, the fluid communication between the first chamber and the second chamber is interruptible. It is understood that the single-use container can comprise one or more valves configured to interrupt the fluid communication between the first chamber and the second chamber.

In still further aspects, at least the first chamber is thermally conductive or inductively heated. In yet still further aspects, at least a portion of the anvil is thermally conductive or inductively heated. In yet still further aspects, at least the first chamber is cooled. In yet still further aspects, at least a portion of the anvil is cooled.

In still further aspects, at least a portion of the single-use container is recyclable. It is understood that any portions of the single-use container can be recycled. For example, the flow-directing unit can be recyclable. In yet other aspects, the first chamber material can be recyclable.

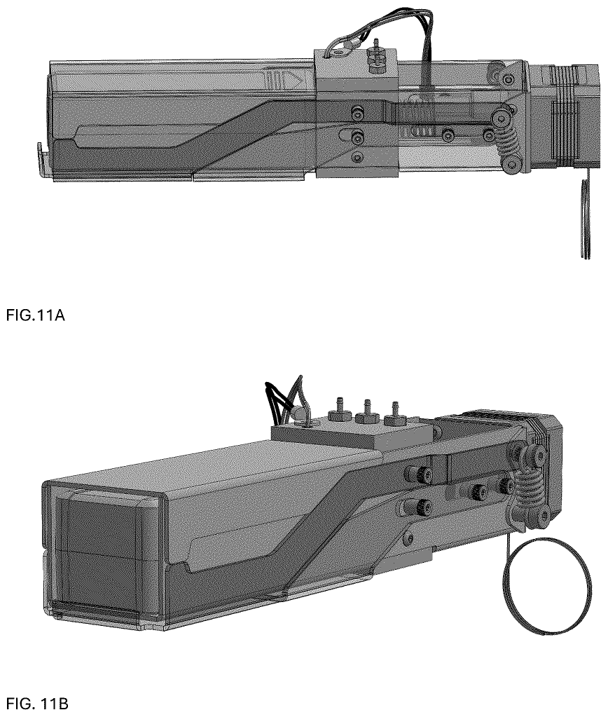

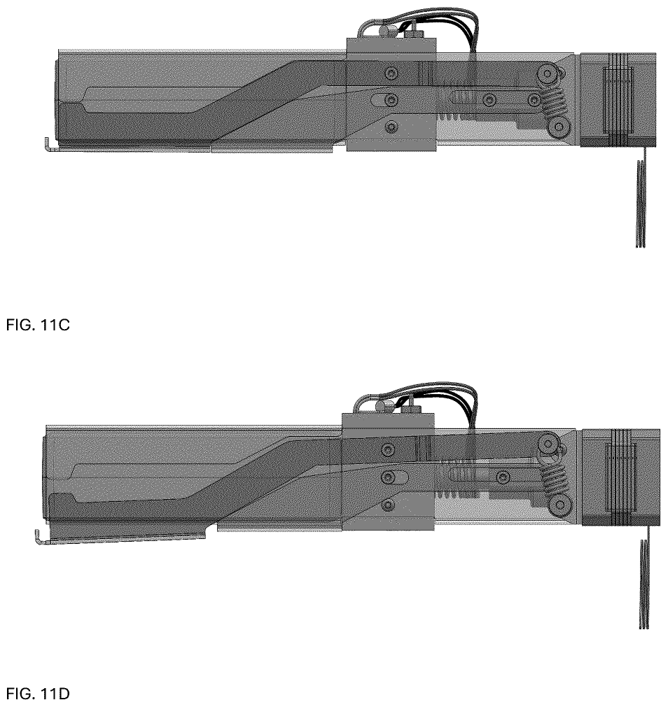

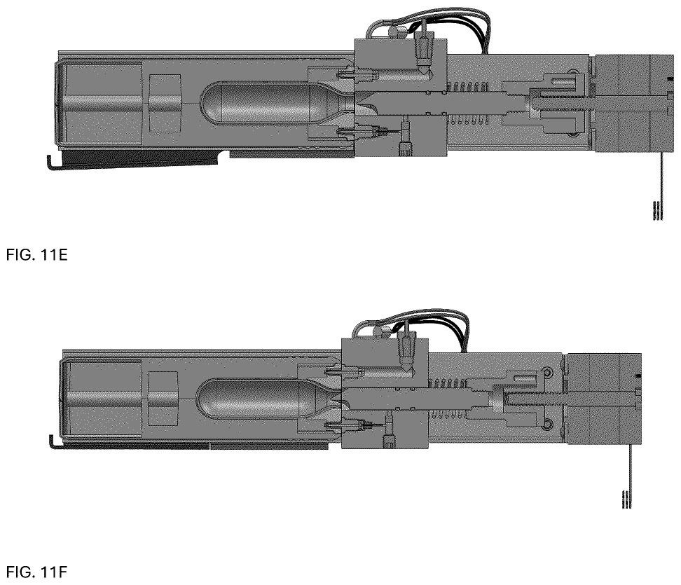

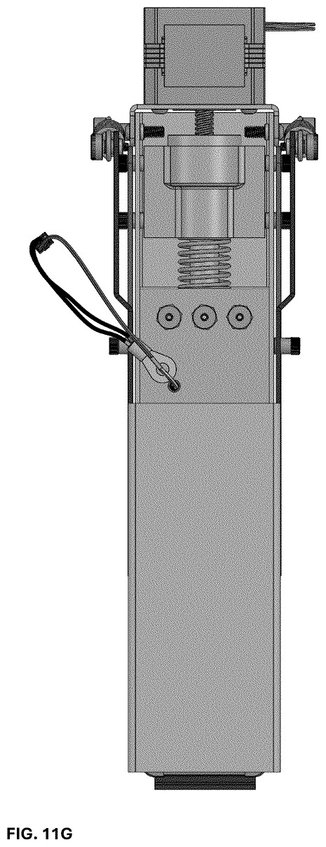

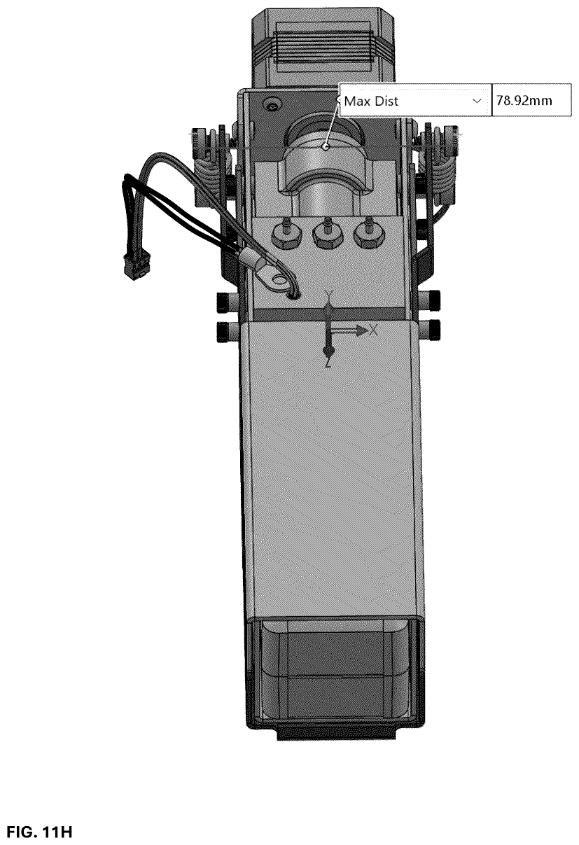

In still further aspects, the single-use container further comprises a locking mechanism configured to lock the single-use container within the apparatus. Such exemplary and unlimiting mechanisms are shown in FIGS. 11A-11J.

In still further aspects, the housing of the single-use container can further comprise a cooling element. In such exemplary and unlimiting aspects, at least the first chamber is in fluid communication with a cooling element. Yet, in other aspects, the cooling element can be positioned within the flow-directing unit. While in still other aspects, the cooling element can be positioned within the apparatus.

In still further aspects, the apparatus can comprise a control unit. In such exemplary and unlimiting aspects, the control unit is configured to engage the anvil with the single-use container.

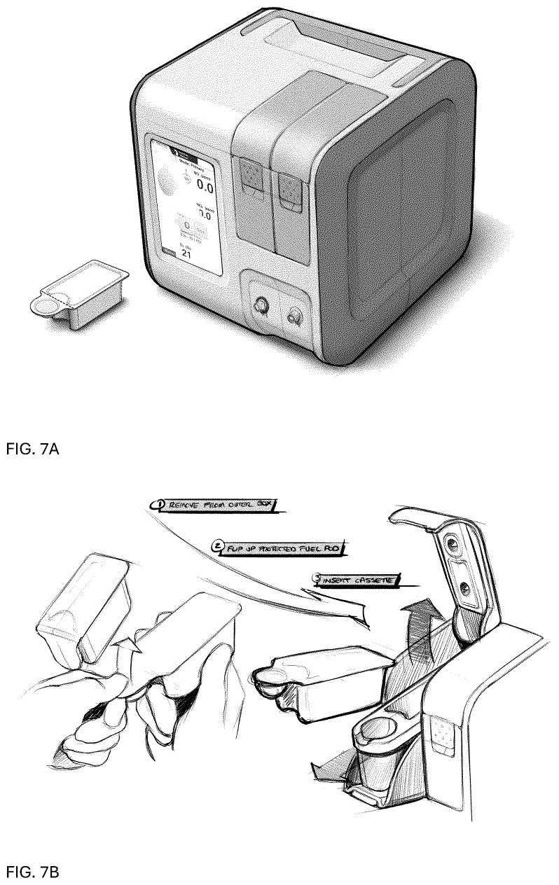

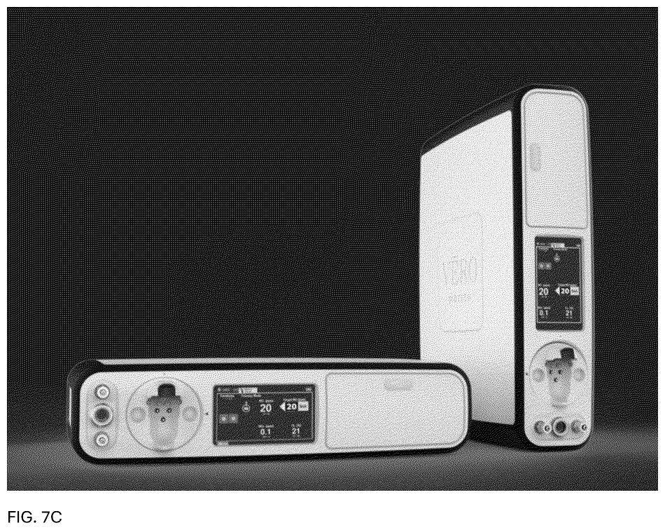

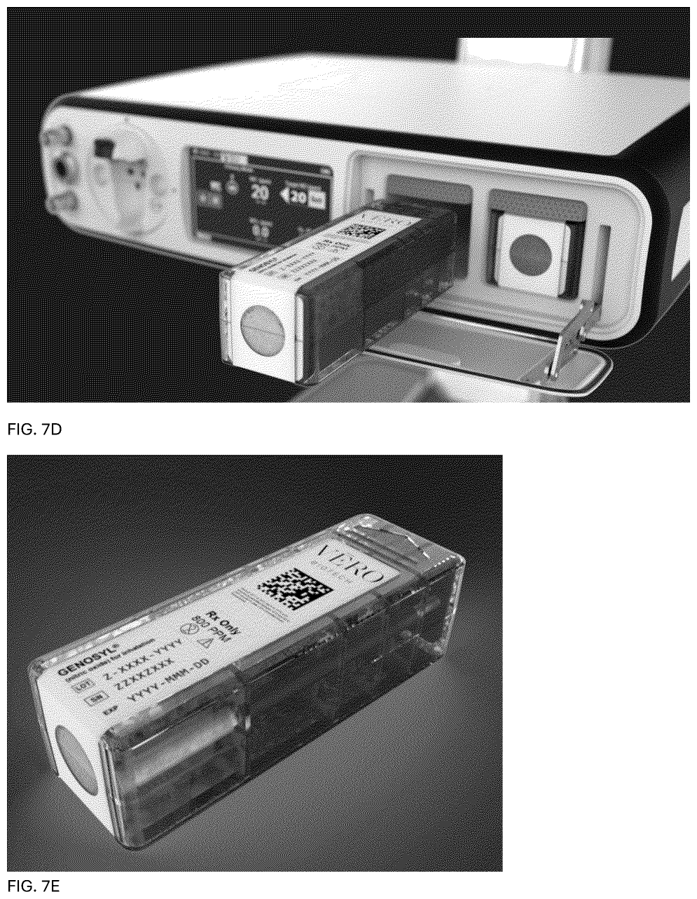

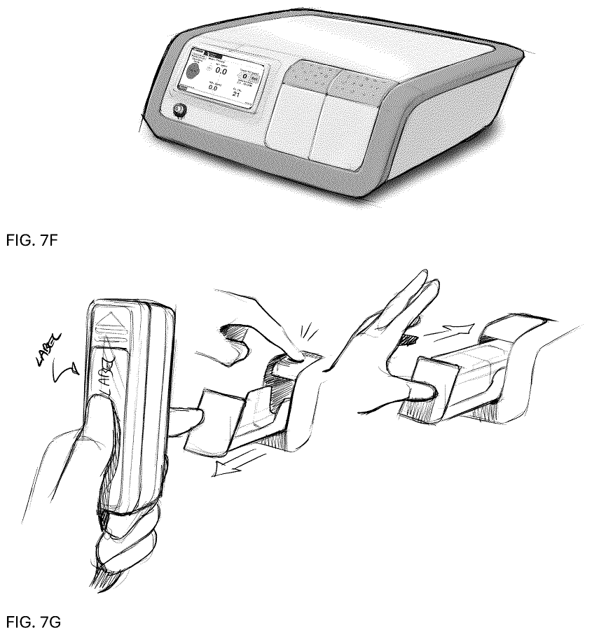

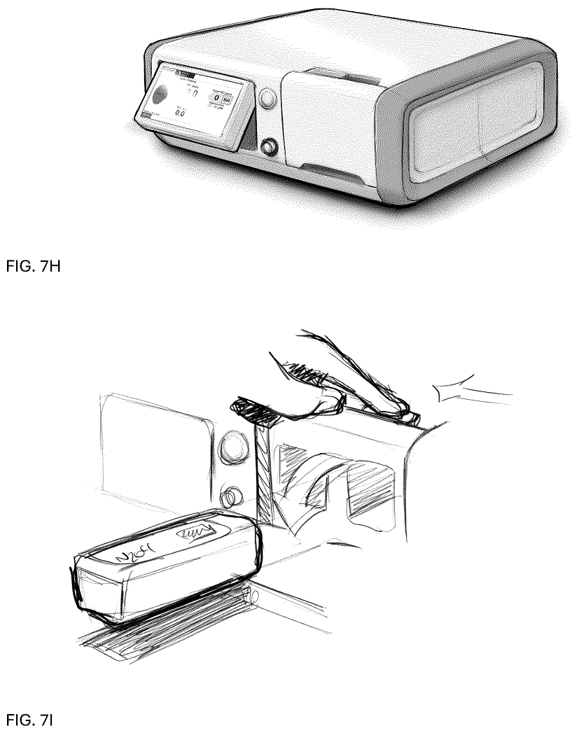

Some exemplary and unlimiting configurations of the single-use containers and apparatuses are shown in FIGS. 7A-7I.

Additional exemplary single-use containers are shown in FIGS. 20A-35 and are described in detail herein.

FIGS. 20A-20C show portions of an exemplary single-use container 2000. More specifically, the figures show exemplary designs for the first chamber in 2010 and the flow-directing unit in 2020.

The first chamber in this example, similar to the examples provided above and below, is configured to be directly filled with N₂O₄ or capable of caring for an ampule comprising N₂O₄. In such aspects, if the chamber is directly filled with N₂O₄, it can be sealed by any known in the art methods. For example, and without limitations, it can be sealed with a metal burst disc. If the chamber contains an ampule, the ampule is conventionally sealed. It is understood that the ampule can be made of glass or any other appropriate material that can withstand the operational conditions of the single-use container.

The first chamber, as described above, in certain aspects, can be constructed of stainless steel, aluminum, a metal alloy, or other thermally conductive metal to enable heating of the N₂O₄/NO₂. Yet in other aspects, the first chamber can be constructed of an inductive metal, which enables inductive heating of the N₂O₄/NO₂. Still, in further aspects, the first chamber can be constructed of a polymer or other suitable material to contain pressure wherein the material is thermally conductive (e.g., thermally conductive polymer). Still, in further aspects, the first chamber can be constructed of any non-conductive or insulating material but contain an inductive target inside, which is in contact with the N₂O₄ and can be heated inductively through the first chamber walls and subsequently transfer its heat to N₂O₄.

FIG. 20A shows the flow-directing unit 2020, having a valve, for example, a spool valve, 2021, positioned within a bed (not shown), similar to the one shown in FIG. 14A (1030). In still further aspects, the bed is substantially smooth to minimize the friction between the valve and the bed.

The distal end of the spool valve 2021 is defined by the sharp edge of a needle 2023. Any known in the art needles capable of unsealing the first chamber and/or ampule can be used. Some exemplary configurations of the needles are shown in FIG. 24. In certain aspects, the needle is a beveled needle. Yet, in other aspects, the needle comprises a least one orifice serving as a vent. In still further aspects, the at least one orifice is positioned on the side of the needle. In such an aspect, the orifice can provide a second flow path through the needle in case the flap covers the main opening in the tip after piercing. It also allows gas to escape to provide a more uniform and repeatable piercing.

In this exemplary aspect, the first chamber comprises a burst disc 2025 that can be unsealed by the described needle. It was found that the positioning of the burst disc within the flow-directing unit and not between the flow-directing unit and the first chamber is beneficial for the device's operation. The flow-directing unit receives the first chamber within the receiving portion 2080 and allows a more convenient filling and sealing of the first chamber, for example, similar to screwing on the cap 2090. It is understood that in such exemplary aspects, the single-use container may comprise an additional internal element ensuring a seal between the burst disc and an internal surface of the flow-directing unit.

In still further aspects, the flow-directing unit 2020 comprises an inlet 2022 and an outlet 2024 that are configured to mate with the appropriate portions of the anvil and are unsealed when the single-use container is inserted into the apparatus. The inlet 2022 and outlet 2024 can comprise a valve 2026 that helps direct the gas flow from the apparatus to the single-use container and from the single-use container to the apparatus. In certain aspects, the valve can be a duckbill valve.

In still further aspects, the flow-directing unit can comprise a micro-orifice plate 2027 similar to the one disclosed above. FIG. 20B shows a side perspective of the cross-section shown in FIG. 20A. FIG. 20C shows a close perspective of the first chamber and the flow-directing until the assembly. The outer shape of the first chamber and the flow-directing unit is substantially symmetrical, which makes it easier to seal the flow-directing unit within the outer body halves and to separate the second and inerting chambers. This simple shape also makes it possible to produce just one molded polymer matrix component and component and install two copies.

FIGS. 21A-21B show a schematic view of the single-use container 2010 containing the first chamber 2110 and the flow-directing unit 2130, similar to those shown in FIGS. 20A-20C. FIG. 21A shows a view of the single-use container with a top portion being removed. The single-use container 2100 further comprises the second chamber 2120 and the inerting chamber 2140. The darker shade (here is a blue shade) lines represent sealing faces between the top (not shown) and the bottom 2190 (FIG. 21B) portions of the outer housing. When the single-use container is activated to form NO from N₂O₄, the N₂O₄ and/or NO₂ flow is contained within the first and the second chambers. However, when N₂O₄ and/or NO₂ are diverted to the inerting flow paths, it flows through the channels on either side of the enclosure and into the inerting chamber 2140. In certain aspects, additional flow-directing elements can be positioned within the second chamber. Such flow-directing elements can comprise, for example, baffles that help in directing the gas flow during the formation of nitric oxide (NO).

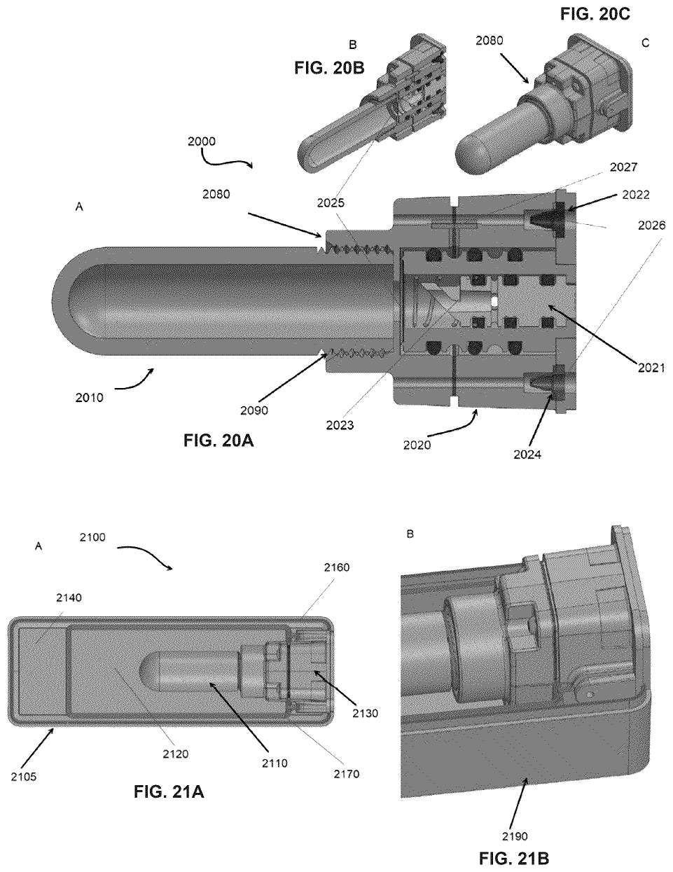

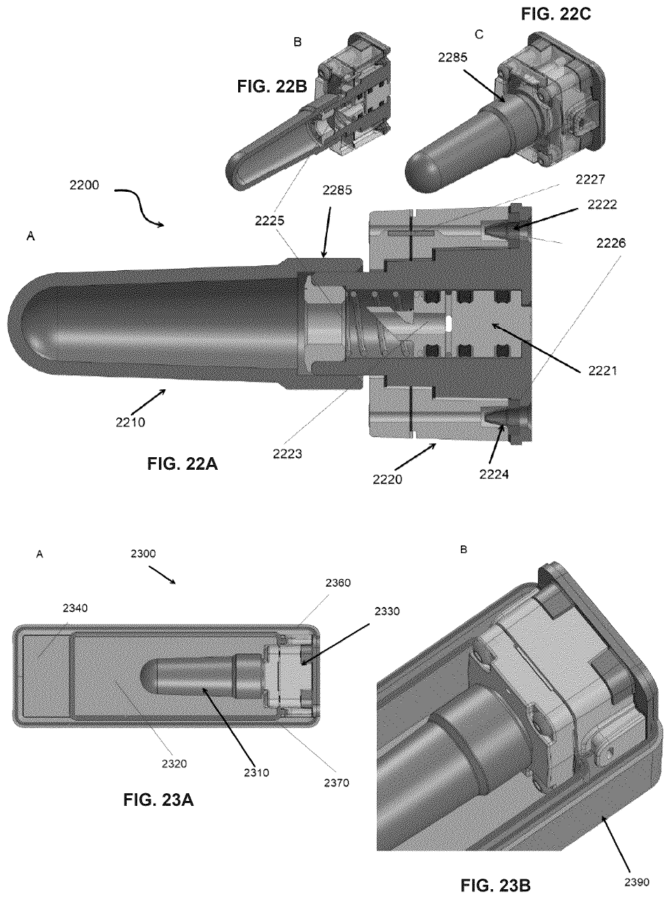

Referring to FIGS. 22A-22C. These figures describe an additional exemplary aspect of the current disclosure. The main components of the configuration shown in FIGS. 22A-22C are similar to those shown in FIGS. 20A-20C: the first chamber 2210, the flow-directing unit in 2220, the needle 2223, the inlet 2222, the outlet 2224, (where the inlet and outlet can comprise a valve 2226, such as a duckbill or other resealable valve. The burst disc 2225 is contained within the flow-directing unit but can retained distally by a threaded cap 2285 (that may double as a seal).

It is understood that the valve, for example, 2021, 2221, or any described herein valves within the flow-directing unit, can have a diameter that is suitable for the desired application. It is understood that in some aspects, the diameter of the valve, such as a spool valve, can determine the type and strength of the spring that holds this valve and thus affect the general activation and retraction mechanisms of the valve.

It is understood that any of the configurations disclosed herein without limitations can also comprise additional elements, such as example, O-rings, gaskets, valves, baffles, etc., to ensure better sealings, flow distribution, and the like.

FIGS. 23A-23B shows the views of the single-use container 2300 comprising elements shown in FIGS. 22A-22C. FIGS. 23A-23B show the first chamber 2310, and the flow-directing unit 2330. FIG. 23A shows a view of the single-use container with a top portion being removed. The single-use container 2300 further comprises the second chamber 2320 and the inerting chamber 2340. The darker shade (here is a blue shade) lines represent sealing faces between the top (not shown) and the bottom 2390 (FIG. 23B) portions of the outer housing. When the single-use container is activated to form NO from N₂O₄, the N₂O₄ and/or NO₂ flow is contained within the first and the second chambers. However, when N₂O₄ and/or NO₂ are diverted to the inerting flow paths, it flows through the channels on either side of the enclosure and into the inerting chamber 2140. In certain aspects, additional flow-directing elements can be positioned within the second chamber. Such flow-directing elements can comprise, for example, baffles that help in directing the gas flow during the formation of nitric oxide (NO).

FIG. 23C shows the single-use container 2300, similar to those shown in FIGS. 22A-22C and 23A-23B, wherein the second chamber 2322 contains any of the disclosed antioxidant-containing media and the first chamber 2310 contains, for example, liquid N₂O₄ 2315 and a gas space 2317. In aspects where the burst disc is pierced, some of the liquid N₂O₄ can be disposed of on the other side of the burst disc.

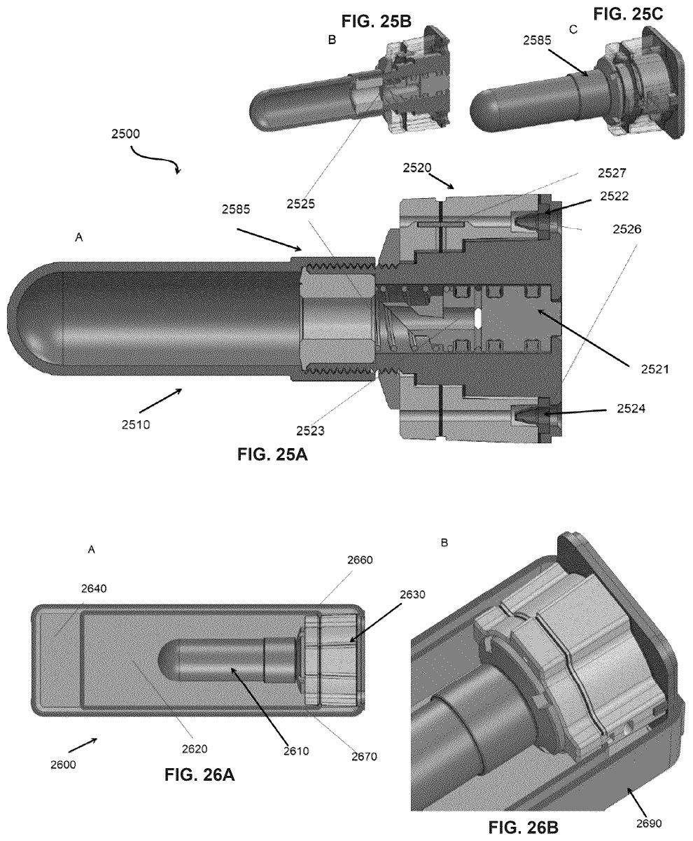

Referring to FIGS. 25A-25C. These figures describe an additional exemplary aspect of the current disclosure. The main components of the configuration shown in FIGS. 25A-25C are similar to those shown in FIGS. 20A-20C and 22A-22C: the first chamber 2510, the flow-directing unit in 2520, the needle 2523, the inlet 2522, the outlet 2524, (where the inlet and outlet can comprise a valve 2526, such as a duckbill valve. The burst disc 2525 is contained within the flow-directing unit but can retained distally by a threaded cap 2585 (that may double as a seal).

Similarly to other configurations described herein, the outer portions of the flow-directing unit can be produced from any known in the art materials. For example, it can be made from metal, plastic materials, or any combination thereof.

Further, in this embodiment, the press-fit cap that secures the burst disc and spool has a sufficiently small inner diameter to accept a first chamber with an externally threaded end.

FIGS. 26A-26B shows the views of the single-use container 2600 comprising elements shown in FIGS. 25A-25C. FIGS. 26A-26B show the first chamber 2610, and the flow-directing unit 2630. FIG. 26A shows a view of the single-use container with a top portion being removed. The single-use container 2600 further comprises the second chamber 2620 and the inerting chamber 2640. The darker shade (here is a blue shade) lines represent sealing faces between the top (not shown) and the bottom 2690 (FIG. 26B) portions of the outer housing. When the single-use container is activated to form NO from N₂O₄, the N₂O₄ flow is contained within the first and the second chambers. However, when N₂O₄ is diverted to the inerting flow paths, it flows through the channels on either side of the enclosure and into the inerting chamber 2140. In certain aspects, additional flow-directing elements can be positioned within the second chamber. Such flow-directing elements can comprise, for example, baffles that help in directing the gas flow during the formation of nitric oxide (NO).

FIG. 26C shows a general schematic of the single-use container 2600 shown in FIGS. 25A-26B, comprising a bottom portion of the housing 2690, a top portion 2694, the first chamber 2610, the second chamber 2620, the flow-directing unit 2630, and the inerting chamber 2640.

FIG. 26D shows a schematic assembly of the single-use container 2600. The bottom portion 2690 of the housing hosts the second chamber 2620, which is shown here schematically as comprising two-molded parts 2620a and 2620b comprising antioxidant-containing media. The molded antioxidant-containing media 2620a and 2620b can be made to include a space to receive the first chamber 2610. The top portion 2695 covers the housing. In certain aspects, the single-use container can further comprise a label or any other identifying element. It is understood that the labels or any other identifying elements can carry information for the caregiver or the patient with respect to content, use, dosage, precautions, and the like as they relate to the single-use container. The exemplary fully assembled single-use container is shown in the FIG. 26E.

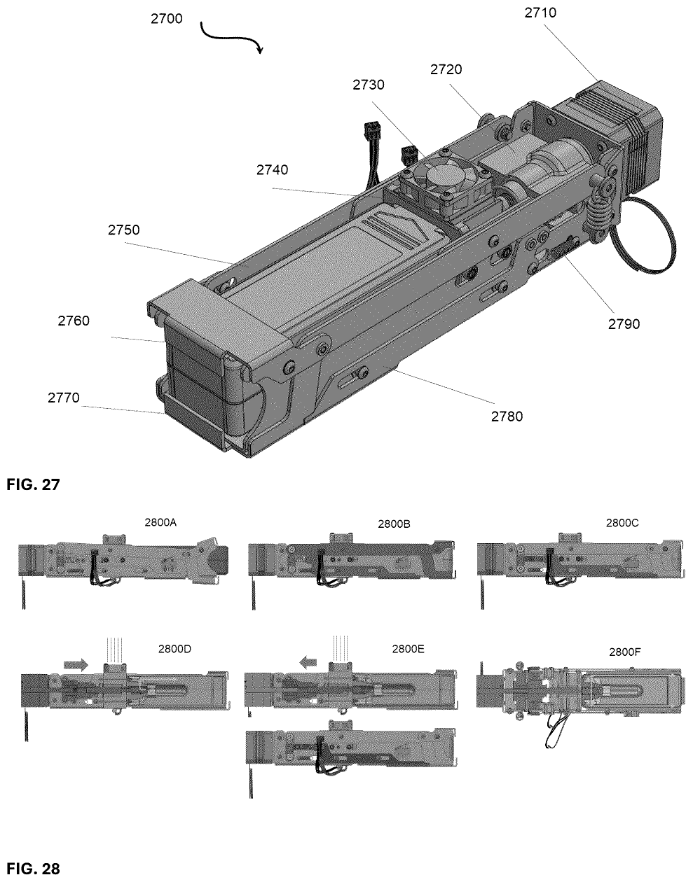

FIG. 27 shows the mechanism 2700 that can be used to activate the single-use container similar to the one shown in FIGS. 25A-26E.

Some exemplary portions of the mechanism are as follows. Heat can be transferred by contact between the anvil and the proximal face of the single-use container, which is also the proximal end of the flow-directing unit. The anvil is heated using a cartridge heater or other means, and the temperature is controlled through a feedback loop using a temperature probe such as a thermistor or thermocouple.

The anvil can be cooled using a fan that blows across or exhausts heat from heat transfer fins, which are part of the anvil. Cooling can also be achieved using a Peltier cooling or similar active cooling device placed in contact with the anvil.

The anvil can contain two valve-piercing or valve-opening elements, which, upon insertion of the single-use container into the mechanism, open flow paths for gas flow into the single-use container and for gas flow out of the single-use container. This is achieved by the elements either opening duck-bill or similar one-way type valves upon insertion, or by the elements having sharp needle end features that pierce a seal. The conveying gas flow into the single-use container can comprise air, oxygen-enriched air, nitrogen, or a combination thereof, and the flow out of the single-use container contains this conveying gas and the formed nitric oxide.

In still further aspects, the mechanism contains a firing pin that contacts and translates a valve with the needle end and side port to different positions to open and close fluid paths as well as piercing a burst disc (or a foil) or break a glass ampule to unseal the first chamber or unseal an ampule containing N₂O₄ into the first chamber.

In yet still further aspects, the mechanism can secure the single-use container once inserted using a two-stage process—when the single-use container is inserted by the user, there are upper and lower jaws that secure it in place once it reaches a certain point of insertion—at this point, the single-use container can still be ejected by depressing the eject lever because it is not yet activated—once the user commands the unit to start dosing, a locking plate is advanced forward which then prevents the upper and lower jaws from being able to open. Once the user is done using the single-use container and commands an ejection, the unit will be cooled, and the firing pin retracts first to an inerting position where the first chamber is fluidically connected to the inerting chamber and then retracts to a home position, which allows the locking plate to return to its home position that allows the user to depress the eject lever and remove the single-use container.

Some of the disclosed above elements are shown in FIG. 27-28 or 31. This configuration utilizes a stepper motor 2710 configured to push both the heater block 2740 and a firing pin 2720 against and into the flow-directing unit (not shown). The heater block is heated and monitored with heating devices (not shown). The mechanism can further comprise a cooling fan 2730 designed to further control the temperature within the first chamber to provide more responsive dose control. Cooling of the heater block is aided by the presence of fins, which increase the available surface area for heat transfer.

The mechanism further can comprise a locking plate 2780, the single-use container lock upper jaw 2760 and lower jaw 2770 (configured to lock the single-use container), and the sheet metal chassis (that forms the structure of the mechanism to hold the single-use container) 2750.

FIG. 28 shows exemplary and unlimiting steps of activation of the single-use container. In step 2800A, the user inserts the single-use container into an apparatus, temporarily opening the sheet metal jaw and closing the limit switch (opening its circuit).

In step 2800B, the single-use container seats itself in the carriage against the heater block and allows the jaw to open the limit switch.

In step 2800C, the limit switch is open (circuit closed), and the stepper motor can drive the firing pin and locking plate forward until the locking plate is stopped by the lower jaw, preventing it from opening during therapy.

In step 2800D, which shows a section view, when the thermistor detects that the heater block has reached the target temperature, the stepper motor can be activated. It travels forward to puncture the burst disc, opens the path to the second chamber, and stops. The air begins to flow over the micro-orifice plate.

In step 2800E, when therapy is stopped, the heater is deactivated, and the fan is activated to cool the heater block. The stepper motor retracts partially to allow the spool valve to direct gas into the inerting chamber (step 2800F), but not far enough to allow the locking plate to slide clear of the lower jaws.

In step 2800F, which shows a top section view, when the firing pin is retracted, the spring returns the spool valve to its starting position, allowing the remaining gas to vent into the inerting chambers wrapped around both sides of the second chamber. Jaws are still locked until the inerting process is complete.

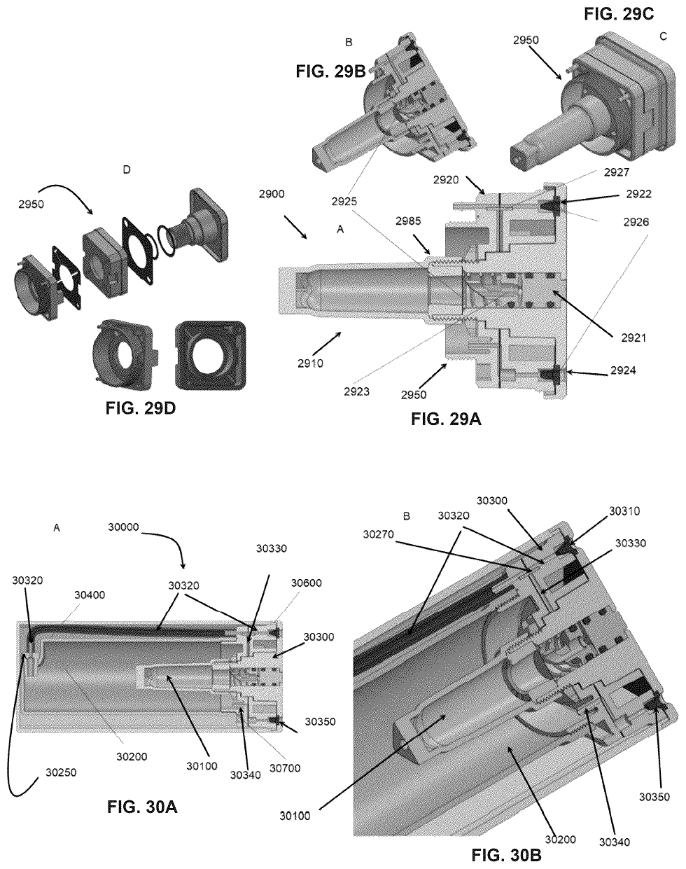

FIGS. 29A-29D show an additional configuration of the first chamber 2910 and the flow-direction unit 2920 that can be used in the single-use container 2900. These figures describe an additional exemplary aspect of the current disclosure. The main components of the configuration shown in FIGS. 29A-29C are similar to those shown in FIGS. 20A-20C, 22A-22C, and FIGS. 25A-25C: the first chamber 2910, the flow-directing unit in 2920, the needle 2923, the inlet 2922, the outlet 2924, (where the inlet and outlet can comprise a valve 2926, such as a Vernay Duckbill Valve. The burst disc 2925 is contained within the flow-directing unit but can retained distally by a threaded cap 2985 (that may double as a seal). An additional threading portion 2950 (the receiving portion for the second chamber to be screwed onto) is shown in FIGS. 29A-29D is directed to receive the second chamber, 30200.

FIGS. 30A-30B show the single-use container 30000 comprising the first chamber 30100 and the flow-directing unit 30300 as shown in FIGS. 29A-29D. 30600 and 30700 show outer and inner seals, respectively. As shown in FIG. 30B, the inlet 30310, and the outlet 30350 are in communication with the second chamber 30200. In this exemplary configuration, the inlet 30310 extends into a first fluidic path 30320 that is configured to deliver a conveying gas provided by the apparatus. It is understood that any of the disclosed above conveying gases can be used. For example, the conveying gas can comprise air, oxygen-enriched air, nitrogen, or any combination thereof.

In still further exemplary aspects, the valve can move such that the flow path 30330 between the first chamber and the second chamber is formed. This flow path delivers the gaseous nitrogen dioxide or the gaseous mixture of dinitrogen tetroxide N₂O₄ and nitrogen dioxide (NO₂) into the first fluidic path 30320 through the micro-orifice plate 30270. In still further aspects, the gaseous nitrogen dioxide, or the gaseous mixture of dinitrogen tetroxide N₂O₄ and nitrogen dioxide (NO₂) are mixed with the conveying gas from the apparatus in the first fluidic path 30320. As can be seen in FIG. 30A, the first fluidic path 30320 is external to the second chamber 30200 and enters the second chamber at a base of a distal portion 30250 of the second chamber. While the first fluidic path in this example is external to the second chamber, it is understood that the aspects where the first fluidic path is internal to the second chamber are also disclosed.

Further, the second chamber comprises a second fluidic path, wherein the second fluidic path 30340 is in flow communication with the outlet 30350 and is configured to deliver formed nitric oxide to the patient.

FIG. 30C shows a schematic of the positioning of the inerting chamber 30400, respectively, to the first chamber 30100, the second chamber 30200, and the flow-directing unit 30300. FIG. 30D shows the assembly of the second chamber 30200, the first chamber 30100, and the flow-directing unit 30300 into the housing 30900.

FIG. 31 shows a mechanism 31000 that can be used to activate the single-use container shown in FIGS. 29A-30D. The mechanism has a stepper motor 31100, firing pin adapter 31300, device heater block 31500, fan 31400, sheet metal chassis 31700, locking plate 31800, single-use container lock spring 31200, locking plate return spring 31600, single-use container lock upper jaw 31200 and lock upper shoe 31300, single-use container lock lower jaw 31100, single-use container receiver tunnel 31900 and eject lever 31400.

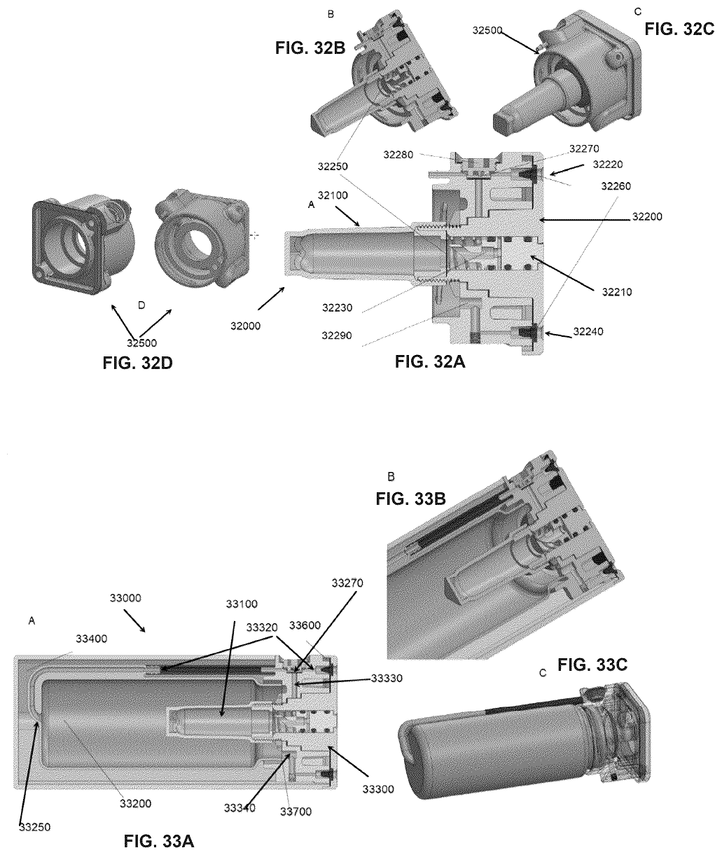

FIGS. 32A-32D show additional exemplary configurations of the first chamber 32100 and the flow-direction unit 32200 that can be used in the single-use container 32000. These figures describe an additional exemplary aspect of the current disclosure. The main components of the configuration shown in FIGS. 32A-32C are similar to those shown in FIGS. 20A-20C, 22A-22C, FIGS. 25A-25C, and FIGS. 29A-29C: the first chamber 32100, the flow-directing unit in 32200, the needle 32230, the inlet 32220, the outlet 32240, (where the inlet and outlet can comprise a valve 32260, such as a duckbill valve. The burst disc 32250 is contained within the flow-directing unit. The flow-directing unit can also comprise an outlet frit receptacle 32390. An additional threading portion 32500, as shown in FIGS. 32A-32D is directed to receive the second chamber 33200.

FIGS. 33A-33C show the single-use container 33000 comprising the first chamber and the flow-directing unit shown in FIGS. 32A-32D. In still further exemplary aspects, the valve can move such that the flow path 33330 between the first chamber 33100 and the second chamber 33200 is formed. This flow path delivers the gaseous nitrogen dioxide or the gaseous mixture of dinitrogen tetroxide N₂O₄ and nitrogen dioxide (NO₂) into the first fluidic path 33320 through the micro-orifice plate 33270. In still further aspects, the gaseous nitrogen dioxide, or the gaseous mixture of dinitrogen tetroxide N₂O₄ and nitrogen dioxide (NO₂), is mixed with the conveying gas from the apparatus in the first fluidic path 33320. As can be seen in FIG. 33A, the first fluidic path 33320 is external to the second chamber 33200 and enters the second chamber at a base of a distal portion 33250 of the second chamber.

In this example, the second chamber 33200 can be an extrusion blow-molded vessel (as shown, for example, in FIG. 33F). The inerting chamber 33400 can be formed by the cavity between the second chamber and the outer body of the single-use container.

Further, the second chamber comprises a second fluidic path, wherein the second fluidic path 33340 is in flow communication with the outlet and is configured to deliver formed nitric oxide to the patient.

FIG. 33B-33E show additional schematics of the single-use cell shown in FIG. 33A.

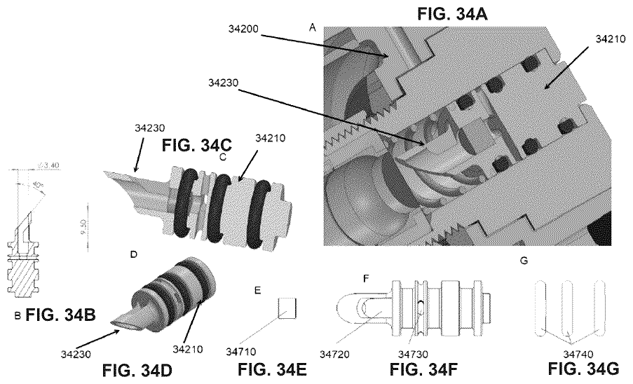

FIGS. 34A-34G show various configurations of the spool valve. The valves can include a steeply beveled needle tip with a side-venting port and an internal lumen. The beveled tip accommodates both foil/disc piercing and ampule shattering when the spool is translated down the axis of the single-use container during activation. The lumen within it connects openings on the face and side of the needle to a plurality of gas passages exiting perpendicular to the axis of the lumen. A small “frit” or filter can be installed in the lumen to prevent the ingestion of glass shards or fluid into these gas passages.

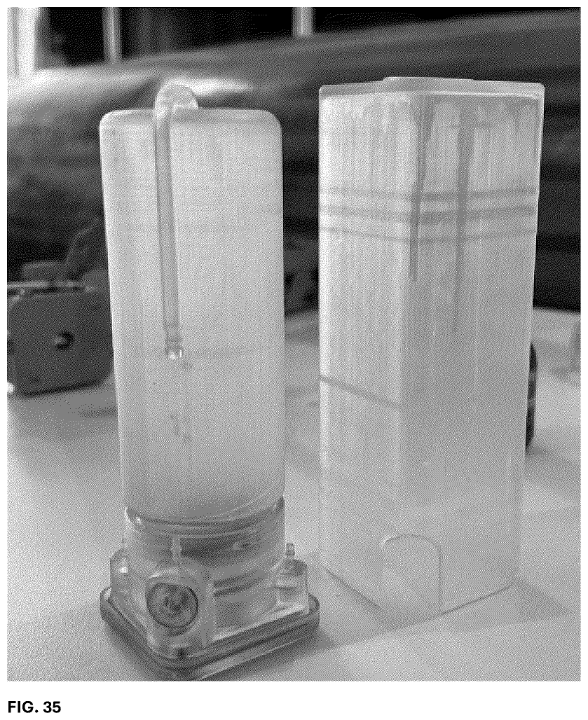

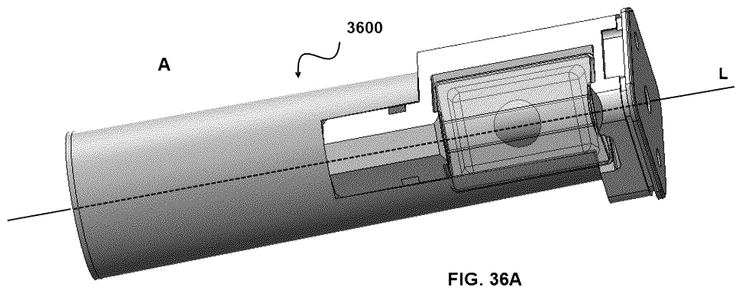

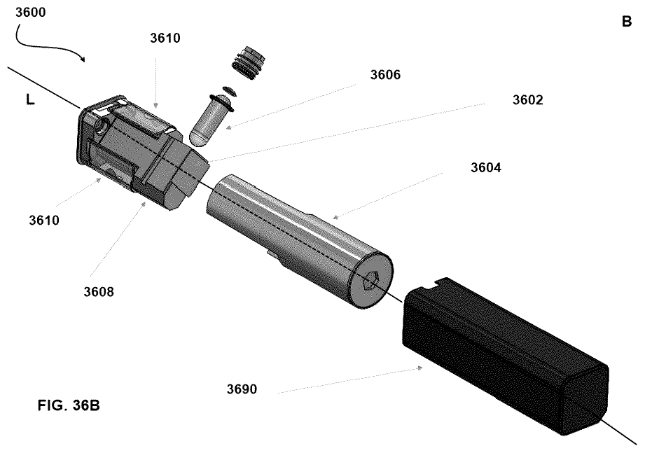

FIG. 35 shows an exemplary single-use container according to some aspects of this disclosure.

An additional aspects of the single-use containers are shown in FIGS. 36A-37B. As discussed above, FIG. 36A shows an assembled version of the exemplary single-use container 3600, where L represents the main longitudinal axis of the container. FIGS. 36B-36F show an exploded view of such a container. In these exemplary and unlimiting aspects, the first chamber 3602 is positioned within the flow-directing unit 3608 and is configured to host an ampule 3606. The ampule 3606 is positioned perpendicular to the main longitudinal axis L (FIG. 36B). The first chamber and the flow-directing units are in communication with the second chamber 3604, and all the parts are hosted within the housing 3690. The two inerting chambers 3610 are positioned within the proximal portion of the single-use container and on the both sides of the flow-directing unit 3608.

FIGS. 36C and 36D show a further exploded view of the container, where more details of the flow-directing unit 3608 are provided. The ampule 3602 is inserted in the first chamber 3602 and is sealed from the surrounding environment by any sealing means 3630 that can include gaskets, O-rings, sealing rings, nuts, bolts, screws, etc. The flow-directing unit 3608 hosts a spool valve 3612, which operates with a spring 3616. Again, all the portions of the flow directing unit, if needed, have additional sealing members 3630 that include seals, O-rings, gaskets, etc. The distal portion of the valve 3612 comprises unsealing means 3614 that are configured to break the ampule 3606 when the container is activated. In certain aspects, the unsealing means 3614 can be releasably attached to the valve 3612. In certain exemplary and unlimiting aspects, when the ampule is unsealed, the unsealing means 3614 can be released from the valve 3612 and stay within the ampule body, thereby preventing undesirable release of broken ampule debris or other particulars to entry the gas flow paths.

In still further aspects, the flow-directing unit can further comprise one or more one-way valves 3640 that help direct the gas flow from the apparatus to the single-use container and from the single-use container to the apparatus. In certain aspects, these one-way valves can be duckbill valves. In still further aspects, these duckbill valves can help the flow-directing unit to direct the gas flow from an inlet to an outlet of the container, as further disclosed below.

In still further aspects, the flow-directing unit can comprise one or more orifices 3672 in which micro-orifices plates 3670 are inserted. Again, all the orifices and openings can be sealed as needed by sealing means 3630 as needed. In FIG. 36D the micro-orifice plates are installed in the holes 3672 and then sealed into place using O-rings and retaining screws (collectively called sealing means 3630). It can be further observed in FIG. 37A. The NO₂ that is formed from N₂O₄ in the first chamber then passes through these micro-orifices, where it is picked up by the conveying gas (blue arrow).

The second chamber 3604 of this exemplary single-use container is shown in FIG. 36F. The antioxidant material 3605 is positioned within the housing and is terminated with end cap 3607.

FIGS. 37A-37B show cutaway views of this exemplary single-use container, and it also show the flow gas paths formed in the container upon activation.

FIG. 37A shows the single-use container 3600 comprising the first chamber 3602 that is positioned within the flow-directing unit 3608. As shown in FIG. 37A, the inlet 3641, and the outlet 3643 are in communication with the second chamber 3604. In this exemplary configuration, the inlet 3641 extends into a first fluidic path 3621 that is configured to deliver a conveying gas provided by the apparatus. It is understood that any of the disclosed above conveying gases can be used. For example, the conveying gas can comprise air, oxygen-enriched air, nitrogen, or any combination thereof.

In still further exemplary aspects, the valve can move such that the flow path 3625 between the first chamber and the second chamber is formed. This flow path delivers the gaseous nitrogen dioxide or the gaseous mixture of dinitrogen tetroxide N₂O₄ and nitrogen dioxide (NO₂) into the first fluidic path 3621 through one or more micro-orifice plates 3670. In still further aspects, the gaseous nitrogen dioxide, or the gaseous mixture of dinitrogen tetroxide N₂O₄ and nitrogen dioxide (NO₂), is mixed with the conveying gas from the apparatus in the first fluidic path 3621. Further, the second chamber comprises a second fluidic path, wherein the second fluidic path 3623 is in flow communication with the outlet 3643 and is configured to deliver formed nitric oxide to the patient.

The one or more micro-orifice plates 3670 can have the same or different sizes. In certain aspects, the one or more micro-orifice plates 3670 have different size that allows for a larger dosing range. The size of the one or more micro-orifices can be 1 micron to 100 microns, including exemplary values of 3, 5, 10, 15, 20, 25, 30, 40, 50, 60, 70, 80 and 90 microns. It is understood that any ranges between any two foregoing values can be formed. For example, and without limitations, the one or more orifices can have a size of 1 to 90 microns, 1 to 70 microns, 1 to 50 microns, 1 to 20 microns, 1 to 10 microns, 5 to 100 microns, 5 to 90 microns, 5 to 50 microns, 5 to 30 microns, and so on. The size of each orifice can assist in determining the flow of nitric oxide as desired. In such aspects, the flow-directing unit can be constructed such that it can accept orifice plates with different orifice sizes—this capability will create the opportunity for single-use containers that support specific dosage ranges—e.g., micro-dosing, standard, high-dose, etc.

In still further aspects, the spool valve 3612 can have various positions. In these exemplary and unlimiting aspects, the spool valve 3612 can have three positions, for example. In one position, the spool valve 3612 breaks the ampule 3606 with the unsealing means 3614 and aligns the gas flow from the ampule to the first micro-orifice plate 3670. In a second position, the valve is configured to align the flow with a second micro-orifice plate 3670. In yet a third position, the valve is configured to align the gas flow with one or more inerting chambers if needed.

In still further aspects, the second chamber 3604 can also comprise a septum 3609. Without wishing to be bound by any theory, it is understood that the septum 3609 is positioned such that it allows the desired direction of the gas flow, as shown with arrows.

FIG. 37B shows a top cutaway view of the single-use container. This view allows one to see the positioning of the one or more inerting chambers 3610 relative to other components (first and second chambers, ampule, valve, etc.) of the single-use container.

It is further understood that this exemplary single-use container can interact with the apparatus similarly to other single-use containers disclosed herein and described above.

It is further understood that the single-use container can be provided in different dosing versions. In such aspects, the different dosing versions of single-use containers can be color-coded and RFID identifiable to distinguish the dosing range and environments of use. Additional possible versions of the single-use container can include high-flow/high-dose and low-flow/low-dose containers, containers for cardiac catheterization lab, standard-dose containers, micro-dose containers, containers for use in neonatal, pediatric, or adult treatment, or containers to provide the treatment during transport of the patient (for example, in the ambulance, medical helicopter, or any other vehicle or aircraft).

In still further aspects, the different versions of the single-use container can be enabled by an interchangeable orifice plate assembly that accepts orifice plates with different hole sizes that allow more or less nitrogen dioxide gas to exit the first chamber as described above.

In still further aspects, disclosed herein is an apparatus comprising: an anvil positioned within a receptacle of the apparatus, wherein the receptacle defines a space configured to receive the single-use container of any of the examples herein, wherein the anvil is engageable with at least the proximal edge of the single-use container, as described in detail above.

In still further aspects, the anvil comprises a first element configured to engage with a first chamber of the single-use container. In such exemplary aspects, the first element is a firing pin configured to unseal the first edge of the valve and to move the valve along the main axis of the single-use container such that the needle unseals the first chamber. In still further aspects, the first element is fluidically connected with an internal lumen of the valve, providing an internal fluidic path connecting the first chamber and the apparatus.

In some exemplary and unlimiting aspects, the anvil can comprise a second element configured to engage with the inlet of the flow-directing unit, such that a first fluidic path connecting the second chamber and the apparatus is formed. In yet still, in further aspects, the anvil can comprise a third element configured to engage with the outlet of the flow-directing unit, such that a second fluidic path connecting the second chamber and the apparatus is formed. In such exemplary and unlimiting aspects, the first fluidic path is directed from the apparatus to the second chamber, and the second fluidic path is directed from the second chamber to the apparatus.

In still further aspects, the apparatus comprises a heating element and/or cooling element. It is understood that the heating element and/or cooling element can be positioned in the anvil, the receptacle of the apparatus, or any combination thereof.

In still further aspects, wherein the internal, first, and/or second fluidic paths comprise a conveying gas comprising air, nitrogen, oxygen, or a combination thereof, wherein the conveying gas is supplied by the apparatus.

In still further aspects, the apparatus disclosed herein can further comprise a control unit. In such exemplary and unlimiting aspects, the control unit is configured to engage the anvil with the single-use container. In yet still further aspects, the anvil can further comprise a temperature measuring device that is in a feedback loop communication with the control unit. In still further aspects, the apparatus is configured to deliver a nitric oxide to a subject.

Also disclosed herein are systems comprising: the single-use container of any of the examples herein, and the apparatus of any of the examples herein.

While various aspects have been described above, it should be understood that they have been presented by way of example only and not limitation. Furthermore, although various aspects have been described as having particular features and/or combinations of components, other aspects possibly have a combination of any features and/or components from any of the aspects, where appropriate, as well as additional features and/or components.

Where methods described above indicate certain events occurring in a certain order, the ordering of certain events may be modified. Additionally, certain of the events may be performed concurrently in a parallel process, when possible, as well as performed sequentially as described above. Although various aspects have been described as having particular features and/or combinations of components, other aspects possibly have a combination of any features and/or components from any of the aspects, where appropriate.

### Exemplary Aspects

### Set A of Exemplary Aspects

Exemplary Aspect 1. A single-use container for forming a therapeutic amount of nitric oxide to be delivered by an apparatus to a subject, wherein the single-use container comprises: a housing defined by a proximal edge and a distal edge and comprising: a first chamber comprising N₂O₄, wherein the first chamber is sealed; a second chamber comprising an antioxidant material; and wherein the first chamber and the second chamber are positioned relative to each other such that when the single-use container is inserted into the apparatus and is activated, the first chamber is unsealed to allow the N₂O₄ to be in fluid communication with the antioxidant material in the second chamber to produce nitric oxide.

Exemplary Aspect 2. The single-use container of any of the examples herein, particularly Exemplary Aspect 1, wherein the second chamber is sealed.

Exemplary Aspect 3. The single-use container of any of the examples herein, particularly Exemplary Aspect 2, wherein the second chamber is unsealed upon insertion into the apparatus.

Exemplary Aspect 4. The single-use container of any of the examples herein, particularly Exemplary Aspects 1-3, wherein the second chamber is positioned concentrically to the first chamber.

Exemplary Aspect 5. The single-use container of any of the examples herein, particularly Exemplary Aspects 1-4, wherein the first chamber is positioned within the second chamber.

Exemplary Aspect 6. The single-use container of any of the examples herein, particularly Exemplary Aspects 1-3, wherein the first chamber and the second chamber are positioned in series.

Exemplary Aspect 7. The single-use container of any of the examples herein, particularly Exemplary Aspects 1-6, wherein, when the unsealing of the first chamber occurs, the formation of the therapeutic amount of nitric oxide is initiated.

Exemplary Aspect 8. The single-use container of any of the examples herein, particularly Exemplary Aspects 1-7, wherein the first chamber is a pressure vessel.

Exemplary Aspect 9. The single-use container of any of the examples herein, particularly Exemplary Aspects 1-8, wherein a distal portion of the housing comprises an inerting chamber.

Exemplary Aspect 10. The single-use container of any of the examples herein, particularly Exemplary Aspects 1-9, wherein the housing comprises an inerting chamber that at least partially encompasses the second chamber.

Exemplary Aspect 11. The single-use container of any of the examples herein, particularly Exemplary Aspect 9 or 10, wherein the inerting chamber comprises an inerting material configured to inert N₂O₄ and/or NO₂.

Exemplary Aspect 12. The single-use container of any of the examples herein, particularly Exemplary Aspects 1-11, wherein the first chamber is directly filled with the N₂O₄ in a liquid form or with the N₂O₄ incorporated within a media.

Exemplary Aspect 13. The single-use container of any of the examples herein, particularly Exemplary Aspects 1-11, wherein the first chamber comprises an ampule filled with the N₂O₄, wherein the ampule is positioned such that a proximal edge of the ampule is aligned with a proximal edge of the first chamber, and wherein the ampule is stationary within the first chamber.

Exemplary Aspect 14. The single-use container of any of the examples herein, particularly Exemplary Aspects 1-13, wherein the antioxidant is dispersed within a media comprising a support material having a specific surface area of 350 to 5000 m²/g.

Exemplary Aspect 15. The single-use container of any of the examples herein, particularly Exemplary Aspects 1-14, wherein the antioxidant comprises ascorbic acid; alpha-, beta-, gamma-, or delta tocopherol; alpha-, beta-, gamma-, or delta-tocotrienol; polyphenols; beta-carotene; or a combination thereof.

Exemplary Aspect 16. The single-use container of any of the examples herein, particularly Exemplary Aspects 1-15, wherein a proximal portion of the housing comprises a flow-directing unit, wherein the flow-directing unit is fluidically connected to the first chamber and the second chamber and is configured to form and/or interrupt a flow path between the first chamber and the second chamber.

Exemplary Aspect 17. The single-use container of any of the examples herein, particularly Exemplary Aspect 16, wherein the flow-directing unit is fluidically connected with the apparatus and is configured to form and/or interrupt a flow path between the first chamber, the second chamber, and the apparatus.

Exemplary Aspect 18. The single-use container of any of the examples herein, particularly Exemplary Aspect 16 or 17, wherein the flow-directing unit is fluidically connected to the inerting chamber and is configured to form and/or interrupt a flow path between the first chamber and the inerting chamber.

Exemplary Aspect 19. The single-use container of any of the examples herein, particularly Exemplary Aspects 16-18, wherein the flow-directing unit comprises a valve comprising a proximal end and a distal end and is configured to move along a main axis of the single-use container and wherein the valve regulates the flow path between the first chamber and the second chamber and/or the first chamber and inerting chamber depending on the valve position.

Exemplary Aspect 20. The single-use container of any of the examples herein, particularly Exemplary Aspect 19, wherein before the single-use container is used, the valve is positioned such that if the first chamber is undesirably unsealed, a flow path is formed between the first chamber and the inerting chamber.

Exemplary Aspect 21. The single-use container of any of the examples herein, particularly Exemplary Aspect 19 or 20, wherein at the end of use of the single-use container, the valve is positioned such that a flow path is formed between the first chamber and the inerting chamber to inert unreacted N₂O₄.

Exemplary Aspect 22. The single-use container of any of the examples herein, particularly Exemplary Aspects 16-21, wherein the valve comprises a needle configured to unseal the first chamber when activated.

Exemplary Aspect 23. The single-use container of any of the examples herein, particularly Exemplary Aspect 22, wherein the needle is a beveled needle.

Exemplary Aspect 24. The single-use container of any of the examples herein, particularly Exemplary Aspect 22 or 23, wherein the needle comprises a least one orifice serving as a vent.

Exemplary Aspect 25. The single-use container of any of the examples herein, particularly Exemplary Aspect 24, wherein at least one orifice is positioned on the side of the needle.

Exemplary Aspect 26. The single-use container of any of the examples herein, particularly Exemplary Aspects 22-25, wherein a sharp edge of the needle defines the distal end of the valve.

Exemplary Aspect 27. The single-use container of any of the examples herein, particularly Exemplary Aspects 22-26, wherein the valve comprises an internal lumen extending from the proximal end to the distal end of the valve and having a first edge and a second edge, wherein the first edge is sealed on the proximal edge of the single-use container before insertion into the apparatus and the second edge defines a distal edge of a sharp edge of the needle.

Exemplary Aspect 28. The single-use container of any of the examples herein, particularly Exemplary Aspect 27, wherein the sharp edge of the needle is open and in fluid communication with the internal lumen of the valve.

Exemplary Aspect 29. The single-use container of any of the examples herein, particularly Exemplary Aspect 27 or 28, wherein the valve comprises one or more orifices that are in fluid communication with the internal lumen and are configured to form one or more further fluid paths.

Exemplary Aspect 30. The single-use container of any of the examples herein, particularly Exemplary Aspects 19-29, wherein the valve further comprises at least one of a frit or a filter.

Exemplary Aspect 31. The single-use container of any of the examples herein, particularly Exemplary Aspects 19-30, wherein the valve is a spring-loaded valve.

Exemplary Aspect 32. The single-use container of any of the examples herein, particularly Exemplary Aspects 16-31, wherein a distal edge portion of the flow-directing unit comprises a mounting space configured to receive the first chamber.

Exemplary Aspect 33. The single-use container of any of the examples herein, particularly Exemplary Aspect 32, wherein the mounting space is configured to be heated.

Exemplary Aspect 34. The single-use container of any of the examples herein, particularly Exemplary Aspects 1-33, wherein the single-use container is configured to be received by a receptacle of the apparatus, wherein at least the proximal edge of the single-use container substantially mates with at least one receiving surface of the receptacle of the apparatus.

Exemplary Aspect 35. The single-use container of Exemplary Aspect 34, wherein the apparatus is configured to activate the single-use container to form nitric oxide.

Exemplary Aspect 36. The single-use container of any of the examples herein, particularly Exemplary Aspects 34-35, wherein when a firing pin of the apparatus activates the valve, the first chamber is unsealed, and a flow path between the first chamber and the second chamber is formed.

Exemplary Aspect 37. The single-use container of any of the examples herein, particularly Exemplary Aspects 34-36, wherein when the first chamber comprises the ampule activation of the valve by a firing pin of the apparatus unseals the first chamber and the ampule to form a flow path between the first chamber, the ampule, and the second chamber.

Exemplary Aspect 38. The single-use container of any of the examples herein, particularly Exemplary Aspects 1-37, wherein the first chamber further comprises a gaseous nitrogen dioxide (NO₂) or a gaseous mixture of dinitrogen tetroxide N₂O₄ and nitrogen dioxide (NO₂).

Exemplary Aspect 39. The single-use container of any of the examples herein, particularly Exemplary Aspect 38, wherein the second chamber is configured to receive the gaseous nitrogen dioxide or the gaseous mixture of dinitrogen tetroxide N₂O₄ and nitrogen dioxide (NO₂) and to form nitric oxide.

Exemplary Aspect 40. The single-use container of any of the examples herein, particularly Exemplary Aspect 38 or 39, wherein the second chamber is configured to transfer the nitric oxide to the subject using the apparatus.

Exemplary Aspect 41. The single-use container of any of the examples herein, particularly Exemplary Aspects 34-40, wherein when the single-use container is positioned within the receptacle, at least a portion of the receiving surface of the receptacle mates with at least a portion of the proximal edge of the housing such that a first edge of the valve is in contact with the firing pin and the valve is moved along a main axis of the single-use container such that sharp end of needle unseals the first chamber and/or ampule if present.

Exemplary Aspect 42. The single-use container of any of the examples herein, particularly Exemplary Aspects 34-41, wherein when the single-use container is positioned within the receptacle, at least a portion of the receiving surface of the receptacle mates with at least a portion of the proximal edge of the housing thereby unsealing an inlet and an outlet in the flow-directing units that are in communication with the second chamber.

Exemplary Aspect 43. The single-use container of any of the examples herein, particularly Exemplary Aspect 42, wherein the inlet extends into a first fluidic path configured to deliver a conveying gas provided by the apparatus.

Exemplary Aspect 44. The single-use container of any of the examples herein, particularly Exemplary Aspect 43, wherein the conveying gas comprises air.

Exemplary Aspect 45. The single-use container of any of the examples herein, particularly Exemplary Aspect 43 or 44, wherein the flow path between the first chamber and the second chamber is configured to deliver the gaseous nitrogen dioxide or the gaseous mixture of dinitrogen tetroxide N₂O₄ and nitrogen dioxide (NO₂) into the first fluidic path.

Exemplary Aspect 46. The single-use container of any of the examples herein, particularly Exemplary Aspect 45, wherein the gaseous nitrogen dioxide or the gaseous mixture of dinitrogen tetroxide N₂O₄ and nitrogen dioxide (NO₂) are mixed with the conveying gas from the apparatus in the first fluidic path.

Exemplary Aspect 47. The single-use container of any of the examples herein, particularly Exemplary Aspects 43-46, wherein the first fluidic path is external to the second chamber and enters the second chamber at a base of a distal portion of the second chamber.

Exemplary Aspect 48. The single-use container of any of the examples herein, particularly Exemplary Aspects 43-46, wherein the first fluidic path is internal within the second chamber.

Exemplary Aspect 49. The single-use container of any of the examples herein, particularly Exemplary Aspects 42-48, wherein the second chamber comprises a second fluidic path, wherein the second fluidic path is in flow communication with the outlet and is configured to deliver formed nitric oxide to the patient.

Exemplary Aspect 50. The single-use container of any of the examples herein, particularly Exemplary Aspects 1-49, wherein the second chamber further comprises flow-enhancing elements configured to provide a uniform flow of the gaseous nitrogen dioxide or the gaseous mixture of dinitrogen tetroxide N₂O₄ and nitrogen dioxide (NO₂), thereby increasing their interaction with the antioxidant.

Exemplary Aspect 51. The single-use container of any of the examples herein, particularly Exemplary Aspect 50, wherein the flow-enhancing elements comprise one or more baffle structures, sieves, gratings, or any combination thereof.

Exemplary Aspect 52. The single-use container of any of the examples herein, particularly Exemplary Aspects 1-51, wherein the fluid communication between the first chamber and the second chamber is interruptible.

Exemplary Aspect 53. The single-use container of any of the examples herein, particularly Exemplary Aspects 1-52, wherein the single-use container comprises one or more valves configured to interrupt the fluid communication between the first chamber and the second chamber.

Exemplary Aspect 54. The single-use container of any of the examples herein, particularly Exemplary Aspects 1-53, wherein at least the first chamber is thermally conductive.

Exemplary Aspect 55. The single-use container of any of the examples herein, particularly Exemplary Aspects 1-54, wherein the first chamber is configured to be inductively heated.

Exemplary Aspect 56. The single-use container of any of the examples herein, particularly Exemplary Aspects 1-55, wherein at least a portion of the single-use container is recyclable.

Exemplary Aspect 57. The single-use container of any of the examples herein, particularly Exemplary Aspects 1-56, wherein the single-use container further comprises a locking mechanism configured to lock the single-use container within the apparatus.

Exemplary Aspect 58. The single-use container of any of the examples herein, particularly Exemplary Aspects 1-57, wherein the apparatus comprises a cooling element such that the cooling element is in thermal communication with at least the first chamber.

Exemplary Aspect 59. The single-use container of any of the examples herein, particularly Exemplary Aspects 1-58, wherein the cooling element is positioned within the flow-directing unit.

Exemplary Aspect 60. An apparatus comprising: an anvil positioned within a receptacle of the apparatus, wherein the receptacle defines a space configured to receive the single-use container of any of the examples herein, particularly Exemplary Aspects 1-59, wherein the anvil is engageable with at least the proximal edge of the single-use container.

Exemplary Aspect 61. The apparatus of any of the examples herein, particularly Exemplary Aspect 60, wherein the anvil comprises a first element configured to engage with a first chamber of the single-use container.

Exemplary Aspect 62. The apparatus of any of the examples herein, particularly Exemplary Aspect 61, wherein the first element is a firing pin configured to unseal the first edge of the valve and to move the valve along a main axis of the single-use container such that the needle unseals the first chamber.

Exemplary Aspect 63. The apparatus of any of the examples herein, particularly Exemplary Aspect 60 or 61, wherein the first element is fluidically connected with an internal lumen of the valve, providing an internal fluidic path connecting the first chamber and the apparatus.

Exemplary Aspect 64. The apparatus of any of the examples herein, particularly Exemplary Aspects 60-63, wherein the anvil comprises a second element configured to engage with the inlet of the flow-directing unit, such that a first fluidic path connecting the second chamber and the apparatus is formed.

Exemplary Aspect 65. The apparatus of any of the examples herein, particularly Exemplary Aspects 60-64, where the anvil comprises a third element configured to engage with the outlet of the flow-directing unit, such that a second fluidic path connecting the second chamber and the apparatus is formed.

Exemplary Aspect 66. The apparatus of any of the examples herein, particularly Exemplary Aspect 64 or 65, wherein the first fluidic path is directed from the apparatus to the second chamber, and the second fluidic path is directed from the second chamber to the apparatus.

Exemplary Aspect 67. The apparatus of any of the examples herein, particularly Exemplary Aspects 60-66, wherein the apparatus comprises a heating element and/or cooling element.

Exemplary Aspect 68. The apparatus of any of the examples herein, particularly Exemplary Aspect 66 or 67, wherein the heating element and/or cooling element are positioned in the anvil, the receptacle of the apparatus, or any combination thereof.

Exemplary Aspect 69. The apparatus of any of the examples herein, particularly Exemplary Aspects 60-68, wherein the internal, first, and/or second fluidic paths comprise a conveying gas comprising air, nitrogen, oxygen, or a combination thereof, wherein the conveying gas is supplied by the apparatus.

Exemplary Aspect 70. The apparatus of any of the examples herein, particularly Exemplary Aspects 60-70, wherein the anvil is movable within the receptacle for the single-use container.

Exemplary Aspect 71. The apparatus of any of the examples herein, particularly Exemplary Aspects 60-70, further comprising a control unit.

Exemplary Aspect 72. The apparatus of any of the examples herein, particularly Exemplary Aspect 71, wherein the control unit is configured to engage the anvil with the single-use container.

Exemplary Aspect 73. The apparatus of any of the examples herein, particularly Exemplary Aspect 70-72, wherein the anvil further comprises a temperature measuring device that is in a feedback loop communication with the control unit.

Exemplary Aspect 74. The apparatus of any of the examples herein, particularly Exemplary Aspects 60-73, wherein the apparatus is configured to deliver a nitric oxide to a subject.

Exemplary Aspect 75. A system comprising: the single-use container of any of the examples herein, particularly Exemplary Aspects 1-59, and the apparatus of any of the examples herein, particularly Exemplary Aspects 60-74.

### Set B of Exemplary Aspects

Example 76. A single-use container for forming a therapeutic amount of nitric oxide to be delivered by an apparatus to a subject, wherein the single-use container comprises: a housing defined by a proximal edge and a distal edge and comprising: a first chamber comprising N₂O₄, wherein the first chamber is sealed; a second chamber comprising an antioxidant material; and wherein the first chamber and the second chamber are positioned relative to each other such that when the single-use container is inserted into the apparatus and is activated, the first chamber is unsealed to allow the N₂O₄ to be in fluid communication with the antioxidant material in the second chamber to produce nitric oxide.

Example 77. The single-use container of any one of the examples herein, particularly Example 76, wherein the first chamber is fully sealed.

Example 78. The single-use container of any one of the examples herein, particularly Example 76, wherein the first chamber is at least partially sealed.

Example 79. The single-use container of any one of the preceding Examples, wherein the second chamber is positioned concentrically to the first chamber.

Example 80 The single-use container of any one of the preceding Examples, wherein the first chamber is positioned within the second chamber.

Example 81. The single-use container of any one of the examples herein, particularly any one of Examples 76-80, wherein the first chamber and the second chamber are positioned in series.

Example 82. The single-use container of any one of the preceding Examples, wherein when the unsealing of the first chamber occurs, formation of the therapeutic amount of nitric oxide is initiated.

Example 83. The single-use container of any one of the preceding Examples, wherein the first chamber is a pressure vessel.

Example 84. The single-use container of any one of the preceding Examples, wherein the housing comprises one or more inerting chambers.

Example 85. The single-use container of any one of the examples herein, particularly Example 84, wherein the one or more inerting chambers are in fluid communication with the first chamber.

Example 86. The single-use container of any one of the examples herein, particularly Example 84, wherein the one or more inerting chambers are positioned in a proximal portion of the housing.

Example 87. The single-use container of any one of the examples herein, particularly Example 85 or 86, wherein the one or more inerting chambers comprise an inerting material configured to inert N₂O₄ and/or NO₂.

Example 88. The single-use container of any one of the examples herein, particularly any one of Examples 76-87, wherein the first chamber comprises an ampule filled with the N₂O₄, and wherein the ampule is stationary within the first chamber.

Example 89. The single-use container of any one of the preceding Examples, wherein the antioxidant material is dispersed within a media comprising a support material having a specific surface area of 350 to 5000 m²/g.

Example 90. The single-use container of any one of the preceding Examples, wherein the antioxidant material comprises ascorbic acid; alpha-, beta-, gamma-, or delta tocopherol; alpha-, beta-, gamma-, or delta-tocotrienol; polyphenols; beta-carotene; or a combination thereof.

Example 91. The single-use container of any one of the preceding Examples, wherein a proximal portion of the housing comprises a flow-directing unit, wherein the flow-directing unit is fluidically connected to the first chamber and the second chamber and is configured to form and/or interrupt a flow path between the first chamber and the second chamber.

Example 92. The single-use container of any one of the examples herein, particularly Example 91, wherein the flow-directing unit is fluidically connected with the apparatus and is configured to form and/or interrupt a flow path between the first chamber, the second chamber, and the apparatus.

Example 93. The single-use container of any one of the examples herein, particularly Example 91 or 92, wherein the flow-directing unit is fluidically connected to the inerting chamber and is configured to form and/or interrupt a flow path between the first chamber and the inerting chamber.

Example 94. The single-use container of any one of the examples herein, particularly any one of Examples 91-93, wherein the flow-directing unit comprises a valve comprising a proximal end and a distal end and is configured to move along a main axis of the single-use container and wherein the valve regulates the flow path between the first chamber and the second chamber and/or the first chamber and inerting chamber depending on a valve position.

Example 95. The single-use container of any one of the examples herein, particularly Example 94, wherein before the single-use container is used, the valve is positioned such that if the first chamber is undesirably unsealed, a flow path is formed between the first chamber and the inerting chamber.

Example 96. The single-use container of any one of the examples herein, particularly Example 94 or 95, wherein at the end use of the single-use container, the valve is positioned such that a flow path is formed between the first chamber and the inerting chamber to inert unreacted N₂O₄ and/or NO₂.

Example 97. The single-use container of any one of the examples herein, particularly any one of Examples 94-96, wherein the ampule is positioned in the first chamber such that it is perpendicular to the valve.

Example 98. The single-use container of any one of the examples herein, particularly Example 97, wherein the distal end of the valve comprises unsealing means to at least partially unseal the ampule when activated, wherein the unsealing means have a proximal end and a distal end, and wherein the distal end of the unsealing means is in contact with at least a portion of the ampule.

Example 99. The single-use container of any one of the examples herein, particularly Example 98, wherein the unsealing means breaks the at least a portion of the ampule when activated.

Example 100. The single-use container of any one of the examples herein, particularly any one of Examples 94-99, wherein the valve comprises an internal lumen extending from the proximal end to the distal end of the valve and having a first edge and a second edge, wherein the first edge is sealed on the proximal edge of the single-use container before insertion into the apparatus and the second edge defines the distal edge of the unsealing means.

Example 101. The single-use container of any one of the examples herein, particularly Example 100, wherein when the ampule is broken, the ampule is in fluid communication with the internal lumen of the valve.

Example 102. The single-use container of any one of the examples herein, particularly any one of Examples 100-101, wherein the valve comprises one or more orifices that are in fluid communication with the internal lumen and are configured to form one or more further fluid paths.

Example 103. The single-use container of any one of the examples herein, particularly any one of Examples 94-102, wherein the valve further comprises at least one of a frit or a filter.

Example 104. The single-use container of any one of the examples herein, particularly any one of Examples 94-103, wherein the valve is a spring-loaded valve.

Example 105. The single-use container of any one of the examples herein, particularly any one of Examples 91-104, wherein the first chamber is positioned within a distal edge portion of the flow-directing unit and is configured to receive the ampule.

Example 106. The single-use container of any one of the examples herein, particularly Example 105, wherein the first chamber is configured to be heated.

Example 107. The single-use container of any one of the examples herein, particularly any one of the preceding Examples, wherein the single-use container is configured to be received by a receptacle of the apparatus, wherein at least the proximal edge of the single-use container substantially mates at least one receiving surface of the receptacle of the apparatus.

Example 108. The single-use container of any one of the examples herein, particularly Example 107, wherein the apparatus is configured to activate the single-use container to form nitric oxide.

Example 109. The single-use container of any one of the examples herein, particularly any one of Examples 107-108, wherein when the valve is activated by a firing pin of the apparatus, the ampule in the first chamber is unsealed, and a flow path between the first chamber and the second chamber is formed.

Example 110. The single-use container of any one of the preceding Examples, wherein the first chamber further comprises a gaseous nitrogen dioxide (NO₂) or a gaseous mixture of dinitrogen tetroxide N₂O₄ and nitrogen dioxide (NO₂).

Example 111. The single-use container of any one of the examples herein, particularly Example 110, wherein the second chamber is configured to receive the gaseous nitrogen dioxide or the gaseous mixture of dinitrogen tetroxide N₂O₄ and nitrogen dioxide (NO₂) and to form nitric oxide.

Example 112. The single-use container of any one of the examples herein, particularly Example 110 or 111, wherein the second chamber is configured to transfer the nitric oxide to the subject using the apparatus.

Example 113. The single-use container of any one of the examples herein, particularly any one of Examples 110-112, wherein when the single-use container is positioned within the receptacle, at least a portion of the receiving surface of the receptacle mates with at least a portion of the proximal edge of the housing such that a first edge of the valve is in contact with the firing pin and the valve is moved along a main axis of the single-use container such that the distal edge of the unsealing means breaks the ampule.

Example 114. The single-use container of any one of the examples herein, particularly any one of Examples 110-113, wherein when the single-use container is positioned within the receptacle, at least a portion of the receiving surface of the receptacle mates with at least a portion of the proximal edge of the housing, thereby unsealing an inlet and an outlet in the flow-directing units that are in communication with the second chamber.

Example 115. The single-use container of any one of the examples herein, particularly Example 114, wherein the inlet extends into a first fluidic path configured to deliver a conveying gas provided by the apparatus.

Example 116. The single-use container of any one of the examples herein, particularly Example 115, wherein the conveying gas comprises air.

Example 117. The single-use container of any one of the examples herein, particularly Examples 115-116, wherein the first fluidic path comprises one or more micro-orifice plates that are fluidically connected with the internal lumen of the valve.

Example 118. The single-use container of any one of the examples herein, particularly Example 117, wherein the first chamber is fluidically connected with the first fluidic path through the one or more micro-orifice plates.

Example 119. The single-use container of any one of the examples herein, particularly Example 118, wherein the gaseous nitrogen dioxide or the gaseous mixture of dinitrogen tetroxide N₂O₄ and nitrogen dioxide (NO₂) from the first chamber is delivered into the first fluidic path.

Example 120. The single-use container of any one of the examples herein, particularly Example 119, wherein the gaseous nitrogen dioxide or the gaseous mixture of dinitrogen tetroxide N₂O₄ and nitrogen dioxide (NO₂) are mixed with the conveying gas from the apparatus in the first fluidic path.

Example 121. The single-use container of any one of the examples herein, particularly Example 120, wherein the first fluidic path delivers the dinitrogen tetroxide N₂O₄ and nitrogen dioxide (NO₂) mixed with the conveying gas into the second chamber.

Example 122. The single-use container of any one of the examples herein, particularly any one of Examples 115-121, wherein the second chamber comprises a second fluidic path, wherein the second fluidic path is in flow communication with the outlet and is configured to deliver formed nitric oxide to the patient.

Example 123. The single-use container of any one of the preceding Examples, wherein the second chamber further comprises flow-enhancing elements configured to provide a uniform flow of the gaseous nitrogen dioxide or the gaseous mixture of dinitrogen tetroxide N₂O₄ and nitrogen dioxide (NO₂), thereby increasing their interaction with the antioxidant.

Example 124. The single-use container of any one of the examples herein, particularly Example 123, wherein the flow-enhancing elements comprise one or more baffle structures, separators, septum, sieves, gratings, or any combination thereof.

Example 125. The single-use container of any one of the examples herein, particularly any one of Examples 122-124, wherein the second chamber comprises a separator configured to direct a first flow from the first fluidic path downward into the second chamber where nitric oxide is formed, and to direct a second flow comprising the formed nitric oxide and the conveying gas of the second chamber into the second fluidic path.

Example 126. The single-use container of any one of the preceding Examples, wherein the fluid communication between the first chamber and the second chamber is interruptible.

Example 127. The single-use container of any one of the preceding Examples, wherein the single-use container comprises one or more valves configured to interrupt the fluid communication between the first chamber and the second chamber.

Example 128. The single-use container of any one of the preceding Examples, wherein at least the first chamber is thermally conductive.

Example 129. The single-use container of any one of the preceding Examples, wherein the first chamber is configured to be inductively heated.

Example 130 The single-use container of any one of the preceding Examples, wherein at least a portion of the single-use container is recyclable.

Example 131. The single-use container of any one of the preceding Examples, wherein the single-use container further comprises a locking mechanism configured to lock the single-use container within the apparatus.

Example 132. The single-use container of any one of the preceding Examples, wherein the apparatus comprises a cooling element such that the cooling element is in thermal communication with at least the first chamber.

Example 133. The single-use container of any one of the preceding Examples, wherein the cooling element is positioned within the flow-directing unit.

Example 134. An apparatus comprising: an anvil positioned within a receptacle of the apparatus, wherein the receptacle defines a space configured to receive the single-use container of any one of the examples herein, particularly any one of Examples 76-133, wherein the anvil is engageable with at least the proximal edge of the single-use container.

Example 135 The apparatus of any one of the examples herein, particularly Example 134, wherein the anvil comprises a first element configured to engage with a first chamber of the single-use container.

Example 136. The apparatus of any one of the examples herein, particularly Example 135, wherein the first element is a firing pin configured to unseal the first edge of the valve and to move the valve along a main axis of the single-use container such that the unsealing means breaks the ampule.

Example 137. The apparatus of any one of the preceding Examples, wherein the anvil comprises a second element configured to engage with the inlet of the flow-directing unit, such that a first fluidic path connecting the second chamber and the apparatus is formed.

Example 138. The apparatus of any one of the preceding Examples, where the anvil comprises a third element configured to engage with the outlet of the flow-directing unit, such that a second fluidic path connecting the second chamber and the apparatus is formed.

Example 139. The apparatus of any one of the examples herein, particularly any one of Examples 134-138, wherein the first fluidic path is directed from the apparatus to the second chamber, and the second fluidic path is directed from the second chamber to the apparatus.

Example 140. The apparatus of any one of the examples herein, particularly any one of Examples 134-139, wherein the apparatus comprises a heating element and/or cooling element.

Example 141. The apparatus of any one of the examples herein, particularly any one of Examples 139-140, wherein the heating element and/or cooling element are positioned in the anvil, the receptacle of the apparatus, or any combination thereof.

Example 142. The apparatus of any one of the examples herein, particularly any one of Examples 134-141, wherein the internal, first, and/or second fluidic paths comprise a conveying gas comprising air, nitrogen, oxygen, or a combination thereof, wherein the conveying gas is supplied by the apparatus.

Example 143. The apparatus of any one of the examples herein, particularly any one of Examples 134-142, wherein the anvil is movable within the receptacle for the single-use container.

Example 144. The apparatus of any one of the examples herein, particularly any one of Examples 134-143, further comprising a control unit.

Example 145. The apparatus of any one of the examples herein, particularly Example 144, wherein the control unit is configured to engage the anvil with the single-use container.

Example 146. The apparatus of any one of the examples herein, particularly any one of Examples 143-145, wherein the anvil further comprises a temperature measuring device that is in a feedback loop communication with the control unit.

Example 147. The apparatus of any one of the examples herein, particularly any one of Examples 134-146, wherein the apparatus is configured to deliver a nitric oxide to a subject.

Example 148. A system comprising: the single-use container of any one of the examples herein, particularly any one of Examples 76-133, and the apparatus of any one of the examples herein, particularly any one of Examples 134-147.

What is claimed is:

1. A single-use container for forming a therapeutic amount of nitric oxide to be delivered by an apparatus to a subject, wherein the single-use container comprises:
a housing defined by a proximal edge and a distal edge and comprising:
a first chamber comprising N₂O₄, wherein the first chamber is sealed;
a second chamber comprising an antioxidant material; and
wherein the first chamber and the second chamber are positioned relative to each other such that when the single-use container is inserted into the apparatus and is activated, the first chamber is unsealed to allow the N₂O₄ to be in fluid communication with the antioxidant material in the second chamber to produce nitric oxide.

2. The single-use container of claim 1, wherein when the unsealing of the first chamber occurs, formation of the therapeutic amount of nitric oxide is initiated.

3. The single-use container of claim 1, wherein the housing comprises one or more inerting chambers, wherein the one or more inerting chambers are in fluid communication with the first chamber, and wherein the one or more inerting chambers comprise an inerting material configured to inert N₂O₄ and/or NO₂

4. The single-use container of claim 1, wherein the first chamber comprises an ampule filled with the N₂O₄, and wherein the ampule is stationary within the first chamber.

5. The single-use container of claim 1, wherein the antioxidant material is dispersed within a media comprising a support material having a specific surface area of 350 to 5000 m²/g, and wherein the antioxidant material comprises ascorbic acid; alpha-, beta-, gamma-, or delta tocopherol; alpha-, beta-, gamma-, or delta-tocotrienol; polyphenols; beta-carotene; or a combination thereof.

6. The single-use container of claim 1, wherein a proximal portion of the housing comprises a flow-directing unit, wherein the flow-directing unit is fluidically connected to the first chamber and the second chamber and is configured to form and/or interrupt a flow path between the first chamber and the second chamber.

7. The single-use container of claim 8, wherein the flow-directing unit is fluidically connected with the apparatus and is configured to form and/or interrupt a flow path between the first chamber, the second chamber, and the apparatus; and/or wherein the flow-directing unit is fluidically connected to the inerting chamber and is configured to form and/or interrupt a flow path between the first chamber and the inerting chamber.

8. The single-use container of claim 7, wherein the flow-directing unit comprises a valve comprising a proximal end and a distal end and is configured to move along a main axis of the single-use container and wherein the valve regulates the flow path between the first chamber and the second chamber and/or the first chamber and inerting chamber depending on a valve position.

9. The single-use container of claim 8, wherein the ampule is positioned in the first chamber such that it is perpendicular to the valve.

10. The single-use container of claim 9 wherein the distal end of the valve comprises unsealing means to at least partially unseal the ampule when activated, wherein the unsealing means have a proximal end and a distal end, and wherein the distal end of the unsealing means is in contact with at least a portion of the ampule, and wherein the unsealing means breaks the at least a portion of the ampule when activated.

11. The single-use container of claim 10, wherein the valve comprises an internal lumen extending from the proximal end to the distal end of the valve and having a first edge and a second edge, wherein the first edge is sealed on the proximal edge of the single-use container before insertion into the apparatus and the second edge defines the distal edge of the unsealing means, and wherein when the ampule is broken, the ampule is in fluid communication with the internal lumen of the valve.

12. The single-use container of claim 11, wherein the valve comprises one or more orifices that are in fluid communication with the internal lumen and are configured to form one or more further fluid paths.

13. The single-use container of claim 8, wherein the valve is a spring-loaded valve.

14. The single-use container of claim 1, wherein the single-use container is configured to be received by a receptacle of the apparatus, wherein at least the proximal edge of the single-use container substantially mates with at least one receiving surface of the receptacle of the apparatus.

15. The single-use container of claim 14, wherein when the valve is activated by a firing pin of the apparatus, the ampule in the first chamber is unsealed, and a flow path between the first chamber and the second chamber is formed.

16. The single-use container of claim 15, wherein the second chamber is configured to receive the gaseous nitrogen dioxide or the gaseous mixture of dinitrogen tetroxide N₂O₄ and nitrogen dioxide (NO₂) and to form nitric oxide.

17. The single-use container of claim 15 wherein the second chamber is configured to transfer the nitric oxide to the subject using the apparatus.

18. The single-use container of claim 14, wherein when the single-use container is positioned within the receptacle, at least a portion of the receiving surface of the receptacle mates with at least a portion of the proximal edge of the housing such that a first edge of the valve is in contact with the firing pin and the valve is moved along a main axis of the single-use container such that the distal edge of the unsealing means breaks the ampule.

19. The single-use container of claim 14, wherein when the single-use container is positioned within the receptacle, at least a portion of the receiving surface of the receptacle mates with at least a portion of the proximal edge of the housing, thereby unsealing an inlet and an outlet in the flow-directing units that are in communication with the second chamber.

20. The single-use container of claim 19, wherein the inlet extends into a first fluidic path configured to deliver a conveying gas provided by the apparatus.

21. The single-use container of claim 20, wherein the first fluidic path comprises one or more micro-orifice plates that are fluidically connected with the internal lumen of the valve, and wherein the first chamber is fluidically connected with the first fluidic path through the one or more micro-orifice plates.

22. The single-use container of claim 21, wherein the gaseous nitrogen dioxide or the gaseous mixture of dinitrogen tetroxide N₂O₄ and nitrogen dioxide (NO₂) from the first chamber is delivered into the first fluidic path and wherein the gaseous nitrogen dioxide or the gaseous mixture of dinitrogen tetroxide N₂O₄ and nitrogen dioxide (NO₂) are mixed with the conveying gas from the apparatus in the first fluidic path.

23. The single-use container of claim 22, wherein the first fluidic path delivers the dinitrogen tetroxide N₂O₄ and nitrogen dioxide (NO₂) mixed with the conveying gas into the second chamber.

24. The single-use container of claim 19, wherein the second chamber comprises a second fluidic path, wherein the second fluidic path is in flow communication with the outlet and is configured to deliver formed nitric oxide to the patient.

25. The single-use container claim 24, wherein the second chamber comprises a separator configured to direct a first flow from the first fluidic path downward into the second chamber where nitric oxide is formed, and to direct a second flow comprising the formed nitric oxide and the conveying gas of the second chamber into the second fluidic path.

26. An apparatus comprising:
an anvil positioned within a receptacle of the apparatus, wherein the receptacle defines a space configured to receive the single-use container of claim 1, wherein the anvil is engageable with at least the proximal edge of the single-use container.
